(19) United States
(12) Patent Application Publication
(10) Pub. No.: US 2011/0275664 A1
(43) Pub. Date: Nov. 10, 2011

(54) Method for treating drug and behavioral addictions

(75) Inventors: Kirk W. Johnson, Moraga, CA (US); Linda May Rothblum Watkins, Boulder, CO (US)

(73) Assignees: The Regents of The University of Colorado,a body corporate; MedicNova, Inc.

(21) Appl. No.: 13/064,485

(22) Filed: Mar. 28, 2011

Related U.S. Application Data
(63) Continuation-in-part of application No. 11/527,757, filed on Sep. 26, 2006, now Pat. No. 7,915,285.
(60) Provisional application No. 60/720,568, filed on Sep. 26, 2005.
(60) Provisional application No. 60/810,038, filed on May 31, 2006.

(51) Int. Cl. A61K 31/437; A61P 25/36

(52) U.S. Cl. 514/300

(57) ABSTRACT

The present invention is directed to the use of ibudilast for treating addictions, including drug and behavioral addictions. In particular, ibudilast is used to diminish the dopamine-mediated reward associated with addictions and to treat withdrawal syndromes after discontinuance of addictive drug use or behavior. In addition, methods are provided for preventing or inhibiting relapse in human subjects having a history of methamphetamine addiction or dependence by the administration of an effective amount of ibudilast.

### CROSS-REFERENCE TO RELATED APPLICATION

This application is a continuation-in-part of U.S. Ser. No. 11/527,757, now U.S. Pat. No. 7,915,285, which, in turn, claims under 35 U.S.C. §119(e) the benefit of provisional application 60/720,568, filed Sep. 26, 2005, and provisional application 60/810,038, filed May 31, 2006, which applications are hereby incorporated by reference in their entireties.

### STATEMENT REGARDING FEDERALLY SPONSORED RESEARCH OR DEVELOPMENT

This invention was made with support under NIH Grants DA017670 and DA015642, from the National Institute of Drug Abuse. Accordingly, the United States Government may have certain rights in this invention.

### FIELD OF THE INVENTION

The present invention relates generally to methods for treating drug and behavioral addictions. In particular, the present invention pertains to methods for treating addictions, such as opiate dependence, with ibudilast (also termed AV411 herein) in order to suppress the release of dopamine in the nucleus accumbens, which is associated with the sense of reward subjects experience in response to addictive drugs and behavior. Additionally, ibudilast can be used for treating withdrawal syndromes after discontinuance of addictive drug use or behavior. Ibudilast is specifically shown to relieve opiate withdrawal symptoms and to attenuate opiate-induced brain glial cell activation which may be linked to opiate tolerance and withdrawal phenomena.

### BACKGROUND OF THE INVENTION

The addictiveness of certain drugs and compulsive behaviors is linked to excitation of dopamine mediated reinforcement/reward pathways in the central nervous system (Abbott (2002) Nature 419:872-874; Montague et al. (2004) Nature 431:760-767). Normally dopamine functions to motivate mammals to perform behaviors important for survival, such as eating and sex, but in subjects with addictions, dopamine induces maladaptive behavior. Subjects with addictions feel compelled to use a substance or perform a behavior repeatedly despite experiencing harmful effects. Virtually all drugs of abuse and compulsive behaviors have been shown to increase extracellular dopamine concentrations in the nucleus accumbens of mammals.

Drugs of abuse induce dopamine-mediated dependence characterized by compulsive drug craving and drug seeking behaviors. The World Health Organization (WHO) has classified addictive drugs into nine groups: 1. alcohol, 2. amphetamines, 3. barbiturates, 4. marijuana, 5. cocaine, 6. hallucinogens, 7. khat, 8. opiates, and 9. organic solvents. Dysregulation of dopamine pathways is also associated with compulsive behavioral addictions, such as excessive eating, drinking, smoking, shopping, gambling, sex, and computer use (Comings et al. (2000) Prog. Brain Res. 126:325-341; Comings et al. (1997) 2:44-56; Blum et al. (2000) J. Psychoactive Drugs 32 suppl:i-iv, 1-112; Potenza (2001) Semin. Clin. Neuropsychiatry 6:217-226; Gianoulakis (1998) Alcohol Health Res. World 22:202-210; Bowirrat et al. (2005) Am. J. Med. Genet. B Neuropsychiatr. Genet. 132:29-37; Di Chiara (2005) Physiol. Behay. 86:9-10; Franken et al. (2005) Appetite 45:198-201; Wang et al. (2004) J. Addict Dis. 23:39-53; Aamodt (1998) Nature Med. 4:660; and Koepp et al. (1998) Nature 393:266-268).

In addition, physical and psychological dependence accompanied by withdrawal syndrome is often associated with use of addictive drugs and compulsive behavior. Withdrawal is defined as the appearance of physical and behavioral symptoms upon reduction or cessation of drug use or compulsive behavior. Withdrawal reflects changes occurring in the central nervous system in response to continued use of a substance or repetition of addictive behavior that usurp the normal mechanisms mediating reinforcement and reward of behavior to motivate the addicted individual to continue consuming a drug or repeating compulsive behavior in the face of serious social, legal, physical and professional consequences. Physical symptoms of withdrawal may include intense cravings, irritability, anxiety, dysphoria, restlessness, lack of concentration, lightheadedness, insomnia, tremor, increased hunger and weight gain, yawning, perspiration, lacrimation, rhinorrhoea, dilated pupils, aching of bones, back and muscles, piloerection, hot and cold flashes, nausea, vomiting, diarrhea, weight loss, fever, and increased blood pressure, pulse and respiratory rate.

The management of opioid withdrawal syndrome has long been recognized as an unmet clinical need. Chronic pain afflicts upwards of one in three adults worldwide. Opioid compounds, such as morphine, are frontline therapeutics for the control of chronic pain. Because chronic pain, by definition, persists for many months (and up to the remainder of the patient's life), morphine and like compounds may be given chronically as well. This is a dire problem because opioids induce dependence upon repeated administration, meaning that continuing administration of opioids is required for patients to function normally. When opioids are discontinued, and also during the temporal lag between successive doses of opioids, the patient goes into withdrawal.

Because opioids exert actions in a wide array of brain, spinal cord and bodily tissues, the effects of opioids, and consequent withdrawal symptomologies, are diverse. The signs of withdrawal are generally opposite to the effects of opioids. For example, morphine causes constipation; withdrawal causes diarrhea. Morphine decreases core body temperature, withdrawal raises it. Morphine causes sedation, withdrawal causes agitation. Additional signs of withdrawal include increased pain, dilated pupils, goose pimples, yawning, cramps, muscle aches, restlessness, extreme anxiety, insomnia, nausea and vomiting, sweating, tearing, tachycardia, and increased blood pressure.

Perversely, although pain reduction is the reason that opioids are administered, pain dramatically rebounds during withdrawal such that pain is not only not controlled by the opioids in the area of the original pain complaint, but rather the entire body is now extraordinarily sensitive to touch and temperature stimuli, misinterpreting ordinarily nonpainful stimuli as painful. Light touch becomes painful. Warm and cool become painful. This twist of everyday sensation into threatening pain (along with the other withdrawal symptomology) destroys, on a daily basis, the lives of many millions in the U.S. alone. It creates great suffering in chronic opioid recipients, in patients needing to discontinue opioids, and in recovering drug addicts, whose desire to avoid withdrawal symptoms may prevent them from escaping from illicit drug use.

The problem is compounded by the fact that there is currently no remedy for withdrawal, short of another dose of opioid. As addicts know, another dose of the drug does nothing to solve the problem but instead only masks the problem until the drug yet again wears off. Current approaches to bringing patients and addicts through withdrawal are dire, including “cold turkey”, sedation, and analgesia. “Detoxification” is often induced with naltrexone (an opioid receptor antagonist) under general anaesthesia or benzodiazepine sedation, in a closely monitored environment such as intensive care. Naltrexone induces acute withdrawal, with symptoms that last for about six days. It is only considered for patients in good health. Other currently employed methods to take humans through withdrawal include administration of non-steroidal anti-inflammatory drugs such as paracetamol, anti-emetics such as metoclopramide, anti-diarrheals such as loperamide, diazepam to reduce anxiety and agitation, and clonidine to decrease anxiety, sweating, and changes in heart rate and blood pressure.

In developing an improved treatment for opioid withdrawal it is important to consider that opioids, including morphine, do not just affect neurons. While opioid-responsive neurons in various brain and spinal cord regions suppress pain, lower core body temperature, alter hormone release, etc. (the classical effects of opioids), it has recently been discovered that opioids also affect a non-neuronal cell type called glia (microglia, astrocytes, oligodendrocytes). Morphine and other opioids activate glia. This activation increases with repeated opioid administration, as evidenced by the upregulation of glia-specific activation markers. That such glial activation contributes to morphine tolerance is supported by the finding that co-administering glial inhibitors along with morphine disrupts the development of morphine tolerance. It follows that reduction of glial activation may be useful as a therapeutic approach to disrupting the development of morphine tolerance. Watkins, L. R. et al. (2005) Trends in Neuroscience 28:661-669; Gul, H. et al. (2000) Pain 89:39-45; Johnston, I. N. et al. (2004) J. Neurosci. 24:7353-65; Raghavendra, V. et al. (2002) J. Neurosci 22 (22):9980-89; Raghavendra, V. et al. (2004) Neuropsychopharmacology 29 (2):327-34; Shavit, Y. et al. (2005) Pain 115:50-59; Song, P. and Zhao, Z. Q. (2001) Neurosci. Res. 39:281-86.

Opioid-driven progressive glial activation causes glia to release neuroexcitatory substances, including the proinflammatory cytokines interleukin-1 (IL-1), tumor necrosis factor (TNF), and interleukin-6 (IL-6). These neuroexcitatory substances counteract the pain-relieving actions of opioids, such as morphine, and drive withdrawal symptomology, as demonstrated by experiments involving co-administration or pro- or anti-inflammatory substances along with morphine. For example, injecting IL-1 into the cerebrospinal fluid of mice at a dose having no behavioral effect on its own blocks the analgesic effect of systemic morphine. Similarly, spinal delivery of morphine and IL-1 receptor antagonist (which prevents IL-1 from exerting its effects), or morphine and the anti-inflammatory cytokine IL-10 (which downregulates the production, release and efficacy of proinflammatory cytokines), enhances the magnitude and duration of morphine analgesia. Indeed, if morphine analgesia is established and then allowed to dissipate, potent analgesia can be rapidly reinstated by injecting IL-1 receptor antagonist, suggesting that dissipation of analgesia is caused by the activities of pain-enhancing proinflammatory cytokines rather than dissipation of morphine's analgesic effects.

The activity of other opioids may also be opposed by activation of glia. Studies show that glia and proinflammatory cytokines compromise the analgesic effects of methadone, at least in part, via non-classical opioid receptors (Watkins, L. R. et al. (2005) Trends Neurosci. 28:661-669). These results suggest that glia and proinflammatory cytokines will be involved in methadone withdrawal, and likely withdrawal from other opioids as well. These data also expand the clinical implications of glial activation, since cross-tolerance between opioids may be explained by the activation of the glial pain facilitatory system, which undermines all attempts to treat chronic pain with opioids.

In summary, opioids excite glia, which in turn release neuroexcitatory substances (such as proinflammatory cytokines) that oppose the effects of opioids and create withdrawal symptoms upon cessation of opioid treatment. Compounds that suppress such glial activation would be beneficial novel therapeutics for treatment of opioid withdrawal.

Inhibition of PDE and attenuation of glial activation is also postulated to play a role in methamphetamine relapse and/or reinstatement. For example, Mori et al., have shown that the PDE4 inhibitor, rolipram, suppressed methamphetamine- and morphine-induced hyperlocomotion in mice (Mori et al., 2000). In rats, rolipram dose-dependently inhibited locomotor hyperactivity and rearing induced by methamphetamine (Iyo et al., 1995), reduced behavioral sensitization to methamphetamine (Iyo et al., 1996), and attenuated the discriminative stimulus effects of methamphetamine and morphine (Yan et al., 2006).

Several studies have focused on methamphetamine's effects on inducing microglial activation (e.g., Escubedo et al., 1998; Guilarte et al., 2003; LaVoie et al., 2004; Pubill et al., 2003; Pubill et al., 2002; Thomas et al., 2004a; Thomas et al., 2004b). For instance, minocycline, a tetracycline-type antibiotic and a known inhibitor of microglial activation, and has been reported to attenuate locomotion and striatal extracellular dopamine levels, and to reduce striatal dopamine transporter levels induced by methamphetamine (Zhang et al., 2006). Minocycline also ameliorates methamphetamine-induced impairment of recognition memory and the development of methamphetamine-induced behavioral sensitization (Mizoguchi et al., 2008). In addition, propentofylline a methylxanthine analog that is a PDE inhibitor and glial-cell modulator, has been reported to reduce the level of CPP induced by both methamphetamine and morphine (Narita et al., 2006).

AV411, an inhibitor of phosphodiesteratse activity also reduces the effects of drugs of abuse by increasing the production of anti-inflammatory and nerve growth factors like IL10 or GDNF, respectively. Yan et al., 2007, have observed that partial reduction in the expression of GDNF (through the use of GDNF (+/−) vs wild-type mice) potentiated methamphetamine self-administration, enhanced the motivation to self-administer methamphetamine as determine by break points using progressive ratio schedules, increased vulnerability to drug-primed reinstatement, and prolonged cue-induced reinstatement of extinguished methamphetamine-seeking behavior. Several other studies have reported that GDNF ameliorates methamphetamine-induced neurotoxicity and its reduction exacerbates it (e.g., Boger et al., 2007; Cass, 1996; Cass et al., 2000; Cass et al., 2006; Cass et al., 1999; Melega et al., 2000).

According to the present inventors, therefore, any of AV411's multimodal means of neuroregulation (e.g., its ability to inhibit PDE, to attenuate the activation of glia, or to increase the production of GDNF), or their combination, could be mechanisms through which both stress- and prime-induced methamphetamine reinstatement was reduced.

Accordingly, there remains a need for improved compounds, compositions, and methods of treatment for drug and behavioral addictions. In particular, drugs are needed that attenuate or abolish the dopamine mediated “reward” associated with addicts' cravings and that alleviate symptoms of withdrawal syndromes after discontinuance of drug use or compulsive behavior. There is also a need for drugs that can prevent or inhibit relapse or reinstatement from a psychostimulant, such as methamphetamine in a human subject.

### SUMMARY OF THE INVENTION

These and other embodiments of the subject invention will readily occur to those of skill in the art in view of the disclosure herein.

In one embodiment, therefore, the present invention provides a method for suppressing the release of dopamine in the nucleus accumbens of a subject suffering from a psychostimulant addiction or psychostimulant dependence by administering to the subject an effective amount of ibudilast. The psychostimulant addiction or dependence is an addiction or dependence on a drug selected from the group consisting of an amphetamine, a methylenedioxymethamphetamine, a methamphetamine, and a dextroamphetamine.

Pursuant to an embodiment of the inventive methodology the psychostimulant addiction or dependence is an addiction or dependence to methamphetamine. According to the inventive methodology, ibudilast can be administered systemically or centrally, as multiple therapeutically effective doses, according to a daily dosing regimen or intermittently.

In another embodiment, the present invention provides a method for treating a psychostimulant addiction or dependence by administering to a subject in need thereof a therapeutically effective amount of ibudilast. According to the inventive treatment methodology, the psychostimulant addiction or dependence is an addiction or dependence on a drug selected from the group consisting of an amphetamine, a methamphetamine, a methylenedioxymethamphetamine, and a dextroamphetamine. According to an aspect of the present invention, the psychostimulant addiction or dependence is to the drug methamphetamine.

According to an embodiment of the inventive method ibudilast diminishes or eliminates methamphetamine-related addiction or methamphetamine-related dependence behavior in a subject. For example, ibudilast diminishes or eliminates methamphetamine-related addictive behavior cues or symptoms of withdrawal syndrome in the subject. Thus, according to the inventive methodology, ibudilast diminishes or eliminates activation of glial cells, astrocytes, or microglia and increases in interleukin-1 expression in the subject suffering from methamphetamine-related addiction or dependence. According to another embodiment, treatment for methamphetamine-related addiction or dependence further comprising administering one or more agents other than ibudilast.

In a specific embodiment, a method is provided for inhibiting relapse of methamphetamine addiction or dependence in a human subject comprising administering to human subject having a history of methamphetamine addiction or dependence an effective amount of ibudilast. A human subject having a history of methamphetamine addiction or dependence is at risk for clinical relapse, which may be induced by any number of factors or situations. In particular, a relapse might be induced by stress, anxiety, surroundings, recontact, or exposure to the drug. In one embodiment, ibudilast inhibits in the human subject methamphetamine-related dependence behavior selected from conditioned place preference, sensitization, or tolerance. The methods provided include co-administration of ibudilast with one or more agents other than ibudilast for treating stress-induced, anxiety-induced, induced by an episodic exposure to methamphetamine, or withdrawal-induced methamphetamine relapse. The method of inhibiting relapse in a human subject applies to the inhibition of relapse of psychostimulant addiction or dependence, in general, including addiction to or dependence on amphetamine, a methylenedioxymethamphetamine, a dextroamphetamine and the like. Likewise a method is provided for preventing relapse of psychostimulant addiction or dependence in a human subject having a history of psychostimulant addiction or dependence comprising administering to a human subject in need thereof an effective amount of ibudilast.

In yet another embodiment, a method is provided for preventing reinstatement or relapse of extinguished response in a subject previously reinforced with methamphetamine, comprising administering to the subject an effective amount of ibudilast. According to the inventive method, ibudilast prevents or attenuates relapse or reinstatement due to stress or a prime-induced reinstatement or relapse to methamphetamine. In one embodiment, the inventive method reduces the frequency of reinstatement or relapse to methamphetamine. The inventive method also diminishes or attenuates the magnitude of relapse by administering to a human subject an effective dose of ibudilast.

In one embodiment a therapeutically effective dose of ibudilast is administered to the subject in advance of a methamphetamine prime. Exemplary of therapeutically effective doses of ibudilast are 2.5 mg/kg, 5.0 mg/kg and 7.5 mg/kg. In human subjects, a typical dose ranges from about 10 mg to about 300 mg per day of ibudilast, preferably about 30 mg to about 200 mg per day and, more preferably, about 50 mg to about 100 mg per day.

As noted above, ibudilast attenuates methamphetamine-related reinstatement behavior selected, such as conditioned place preference, sensitization, tolerance and dependence behavior by diminishing or eliminating glial cell activation, or by diminishing or eliminating astrocyte or microglia activation.

The inventive method also provides for a treatment regimen involving the administration of one or more agents other than ibudilast for treating stress-induced or prime-induced methamphetamine reinstatement. Administration of ibudilast or a combination of ibudilast and one or more agents to a subject in need of treatment can be achieved intraperitoneally, intravenously, subcutaneously, orally, intranasally, or sublingually.

### BRIEF DESCRIPTION OF THE FIGURES

FIG. 1 presents pharmacokinetics and tissue distribution for ibudilast in rats.

FIGS. 2A, 2B, 2C and 2D are time courses (in minutes) of withdrawal symptoms (as measured by a total withdrawal score) for various treatment and control protocols in a rat model of morphine withdrawal syndrome.

FIG. 3 shows the levels of dopamine (DA) in the nucleus accumbens (NAc) of rats treated with morphine in the presence and absence of ibudilast (AV411).

FIG. 4 compares the withdrawal behavior during microdialysis of rats which were treated with morphine in the presence and absence of ibudilast (AV411).

FIG. 5 compares naloxone-induced withdrawal behavior of rats treated with morphine and ibudilast at a low dosage (2.5 mg/kg), ibudilast at a high dosage (7.5 mg/kg), or vehicle (PEG).

FIGS. 6A-6C show immunohistochemical analyses of brain samples collected from rats. FIG. 6A shows a brain sample from an animal treated with vehicle and morphine. FIG. 6B shows a brain sample from a naive animal. FIG. 6C shows a brain sample from an animal treated with ibudilast and morphine. Morphine caused significant microglial activation in the periaqueductal grey region as indicated by CD11b staining (FIG. 6A). Treatment with ibudilast dramatically reduced the increase in the CD11b marker caused by chronic morphine administration (FIG. 6C).

FIG. 7 shows a densitometry analysis of the microglial activation marker CD11b from brain samples. Ibudilast caused a significant reduction in the microglial activation marker CD11b in 2 brain regions, the periaqueductal grey and the brain homologue of the spinal dorsal horn, the trigeminal nucleus.

FIG. 8 shows a comparison of IL-1 expression in brain tissue collected from animals treated with ibudilast, ibudilast and morphine, and morphine and vehicle (PEG). Morphine increased IL-1 mRNA in the dorsal but not the ventral periaqueductal grey region of the brain. Ibudilast blocked the morphine induced increase in IL-1 mRNA in the dorsal periaqueductal grey region.

FIG. 9 shows ibudilast-attenuated weight loss in animals experiencing spontaneous opioid withdrawal.

FIG. 10 shows nucleus accumbens dopamine levels in morphine-dependent animals following morphine administration (at time 0) and during naloxone precipitated opioid withdrawal (10 mg/kg of naloxone was administered subcutaneously for 60 minutes) in animals treated with ibudilast (7.5 mg/kg) or vehicle (PEG).

FIG. 11 shows nucleus accumbens dopamine levels in morphine-dependent animals following morphine administration (at time 0) in animals treated with ibudilast (7.5 mg/kg), morphine, or a combination of ibudilast and morphine.

FIG. 12(A) illustrates mean number of active lever presses during foot shock-induced reinstatement testing as a function of AV411 dose. Brackets through the bars indicate ±S.E.M. “VEH” refers to results from vehicle treated group; dashed horizontal lines indicate the range of the means of active lever presses across dosage groups occurring during the last session of extinction. Asterisks (*) indicate a result that is significantly different (p<0.05) from vehicle.

FIG. 12(B) illustrates mean number of inactive lever presses during foot shock-induced reinstatement testing as a function of AV411 dose.

FIG. 13(A) illustrates mean number of active lever presses during methamphetamine prime reinstatement testing as a function of AV411 dose. Brackets through the bars indicate ±S.E.M. “VEH” refers to results from vehicle treated group; dashed horizontal lines indicate the range of the means of active lever presses across dosage groups occurring during the last session of extinction. Asterisks (*) indicate a result that is significantly different (p<0.05) from vehicle.

FIG. 13(B) illustrates mean number of inactive lever presses during methamphetamine prime reinstatement testing as a function of AV411 dose

### DETAILED DESCRIPTION OF THE INVENTION

The practice of the present invention will employ, unless otherwise indicated, conventional methods of chemistry, biochemistry, and pharmacology, within the skill of the art. Such techniques are explained fully in the literature. See, e.g.; A. L. Lehninger, Biochemistry (Worth Publishers, Inc., current addition); Morrison and Boyd, Organic Chemistry (Allyn and Bacon, Inc., current addition); J. March, Advanced Organic Chemistry (McGraw Hill, current addition); Remington: The Science and Practice of Pharmacy, A. Gennaro, Ed., 20^{th} Ed.; Goodman & Gilman The Pharmacological Basis of Therapeutics, J. Griffith Hardman, L. L. Limbird, A. Gilman, 10^{th} Ed.

All publications, patents and patent applications cited herein, whether supra or infra, are hereby incorporated by reference in their entirety.

### I. DEFINITIONS

In describing and claiming the present invention, the following terminology will be used in accordance with the definitions described below.

It must be noted that, as used in this specification and the intended claims, the singular forms “a,” “an,” and “the” include plural referents unless the context clearly dictates otherwise. Thus, for example, reference to “a drug” includes a single drug as well as two or more of the same or different drugs, reference to “an optional excipient” refers to a single optional excipient as well as two or more of the same or different optional excipients, and the like.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, hydrobromide, and nitrate salts, or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Active molecule” or “active agent” as described herein includes any agent, drug, compound, composition of matter or mixture which provides some pharmacologic, often beneficial, effect that can be demonstrated in-vivo or in vitro. This includes foods, food supplements, nutrients, nutriceuticals, drugs, vaccines, antibodies, vitamins, and other beneficial agents. As used herein, the terms further include any physiologically or pharmacologically active substance that produces a localized or systemic effect in a patient.

“Substantially” or “essentially” means nearly totally or completely, for instance, 95% or greater of some given quantity.

“Optional” or “optionally” means that the subsequently described circumstance may or may not occur, so that the description includes instances where the circumstance occurs and instances where it does not.

The term “central nervous system” or “CNS” includes all cells and tissue of the brain and spinal cord of a vertebrate. Thus, the term includes, but is not limited to, neuronal cells, glial cells (astrocytes, microglia, oligodendrocytes), cerebrospinal fluid (CSF), interstitial spaces and the like.

The terms “subject”, “individual” or “patient” are used interchangeably herein and refer to a vertebrate, preferably a mammal. Mammals include, but are not limited to, murines, rodents, simians, humans, farm animals, sport animals and pets.

The term “about”, particularly in reference to a given quantity, is meant to encompass deviations of plus or minus five percent.

The term “addiction” is defined herein as compulsively using a drug or performing a behavior repeatedly that increases extracellular dopamine concentrations in the nucleus accumbens. An addiction may be to a drug including, but not limited to, psychostimulants, narcotic analgesics, alcohols and addictive alkaloids such as nicotine, cannabinoids, or combinations thereof. Exemplary psychostimulants include, but are not limited to, amphetamine, dextroamphetamine, methamphetamine, phenmetrazine, diethylpropion, methylphenidate, cocaine, phencyclidine, methylenedioxymethamphetamine and pharmaceutically acceptable salts thereof. Exemplary narcotic analgesics include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof. Addictive drugs also include central nervous system depressants, such as barbiturates, chlordiazepoxide, and alcohols, such as ethanol, methanol, and isopropyl alcohol. The term addiction also includes behavioral addictions, for example, compulsive eating, drinking, smoking, shopping, gambling, sex, and computer use.

A subject suffering from an addiction experiences addiction-related behavior, cravings to use a substance in the case of a drug addiction or overwhelming urges to repeat a behavior in the case of a behavioral addiction, the inability to stop drug use or compulsive behavior in spite of undesired consequences (e.g., negative impacts on health, personal relationships, and finances, unemployment, or imprisonment), reward/incentive effects associated with dopamine release, salience of drug- or behavior-associated cues, dependency, tolerance, or any combination thereof.

Addiction-related behavior in reference to a drug addiction includes behavior resulting from compulsive use of a drug characterized by dependency on the substance. Symptomatic of the behavior is (i) overwhelming involvement with the use of the drug, (ii) the securing of its supply, and (iii) a high probability of relapse after withdrawal.

The terms “effective amount” or “pharmaceutically effective amount” of a composition or agent, as provided herein, refer to a nontoxic but sufficient amount of the composition to provide the desired response, such as suppression of the release of dopamine in the nucleus accumbens of a subject or suppression of glial activation in a subject, and optionally, a corresponding therapeutic, prophylactic, or inhibitory effect. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation.

The term “extinction” refers to a form of learning in which associations between conditioned drug craving and withdrawal elicited cues and the events they predict are weakened by exposure to the cues in the absence of those events. As applied to animal models of drug addiction and to behavioral correlates in human drug addicts, “extinction” refers to reduced drug-seeking or drug-administration over time.

The term “reinstatement” refers to a preference or aversion induced by a drug prime or by application of a stressful stimulus following extinction training in an in vivo model of clinical relapse. That is, “reinstatement” and “relapse” have some similar connotations in that both involve a period of volitional drug taking, a period of abstinence (forced in reinstatement studies), and renewed drug seeking provoked by classes of determinants (e.g., stress, drug-associated cues—even of an environmental context, and recontact with or an episodic exposure to the drug). However, it would seem that the term reinstatement is more appropriate in a non-clinical laboratory setting involving animal models and the term relapse is more appropriate in a clinical setting involving human subjects.

By “therapeutically effective dose or amount” of ibudilast is intended an amount that, when ibudilast is administered as described herein, brings about a positive therapeutic response in treatment of a drug or behavioral addiction, such as diminishing or eliminating addiction-related behavior of a subject, diminishing or eliminating cravings associated with addiction to a drug or a behavior in a subject, diminishing or eliminating tolerance to a drug in a subject, diminishing or eliminating the incentive salience of drug- or behavior-associated cues in a subject, and/or diminishing or eliminating symptoms of withdrawal caused by reduction or cessation of addictive drug use or behavior by a subject.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery of a novel therapeutic methodology for safely and effectively treating addiction with ibudilast. The methods of the invention reduce the release of dopamine in the nucleus accumbens, which is associated with cravings and compulsive behavior in addicts. The methods of the invention are particularly useful in diminishing or eliminating addiction-related behavior and alleviating symptoms of withdrawal syndromes in a subject.

The present invention also provides a method for treating or attenuating prime-induced or stress-induced methamphetamine relapse in a human subject or reinstatement in an animal model. Methamphetamine can activate glia in vitro and human brain microglial activation has been linked with methamphetamine abuse. Inhibitors or attenuators of glial cell activation, for example, modulators of glial cell activity, such as certain inhibitors of phosphodiesterase activity or the tetracycline-like antibiotic minocycline are candidate therapeutics for preventing and treating psychostimulant (e.g., methamphetamine) relapse.

In order to further an understanding of the invention, a more detailed discussion is provided below regarding methods of treating addictions with ibudilast.

Treatment of Addictions with Ibudilast

Dopamine release in the nucleus accumbens is thought to mediate the “reward” motivating drug use and compulsive behavior associated with addictions. In one aspect, the invention provides a method for suppressing the release of dopamine in the nucleus accumbens of a subject comprising administering to the subject a composition comprising an effective amount of ibudilast.

Ibudilast has been shown in the present application to suppress the release of dopamine in the nucleus accumbens. As shown in Example 3, ibudilast suppresses dopamine release in the nucleus accumbens in rats treated with morphine, as measured by in vivo microdialysis. In addition, ibudilast suppresses naloxone-induced behavioral signs of morphine withdrawal in rats.

Thus, the invention relates to the use of ibudilast to treat addictions, and in particular, to the use of ibudilast to attenuate or abolish the dopamine mediated “reward” associated with addictions, thus diminishing or eliminating cravings associated with addictions and the accompanying addiction-related behavior and withdrawal syndromes of a subject.

In certain embodiments, a therapeutically effective amount of ibudilast can be administered to a subject to treat a drug addiction. The subject can be addicted to one or more drugs including, but not limited to, psychostimulants, narcotic analgesics, alcohols and addictive alkaloids, such as nicotine, cannabinoids, or combinations thereof. Exemplary psychostimulants include, but are not limited to, amphetamine, dextroamphetamine, methamphetamine, phenmetrazine, diethylpropion, methylphenidate, cocaine, phencyclidine, methylenedioxymethamphetamine and pharmaceutically acceptable salts thereof. Exemplary narcotic analgesics include, but are not limited to, alfentanyl, alphaprodine, anileridine, bezitramide, codeine, dihydrocodeine, diphenoxylate, ethylmorphine, fentanyl, heroin, hydrocodone, hydromorphone, isomethadone, levomethorphan, levorphanol, metazocine, methadone, metopon, morphine, opium extracts, opium fluid extracts, powdered opium, granulated opium, raw opium, tincture of opium, oxycodone, oxymorphone, pethidine, phenazocine, piminodine, racemethorphan, racemorphan, thebaine and pharmaceutically acceptable salts thereof. Addictive drugs also include central nervous system depressants, including, but not limited to, barbiturates, chlordiazepoxide, and alcohols, such as ethanol, methanol, and isopropyl alcohol.

In other embodiments, a therapeutically effective amount of ibudilast can be administered to a subject to treat a behavioral addiction. A behavioral addiction can include, but is not limited to, compulsive eating, drinking, smoking, shopping, gambling, sex, and computer use.

In certain embodiments, ibudilast is used in combination therapy with one or more other agents for treating an addiction. Such agents include, but are not limited to, analgesics, NSAIDs, antiemetics, antidiarrheals, alpha-2-antagonists, benzodiazepines, anticonvulsants, antidepressants, and insomnia therapeutics. Exemplary agents include, but are not limited to, buprenorphine, naloxone, methadone, levomethadyl acetate, L-alpha acetylmethadol (LAAM), hydroxyzine, diphenoxylate, atropine, chlordiazepoxide, carbamazepine, mianserin, benzodiazepine, phenoziazine, disulfuram, acamprosate, topiramate, ondansetron, sertraline, bupropion, amantadine, amiloride, isradipine, tiagabine, baclofen, propranolol, desipramine, carbamazepine, valproate, lamotrigine, doxepin, fluoxetine, imipramine, moclobemide, nortriptyline, paroxetine, sertraline, tryptophan, venlafaxine, trazodone, quetiapine, zolpidem, zopiclone, zaleplon, gabapentin, naltrexone, paracetamol, metoclopramide, loperamide, clonidine, lofexidine, and diazepam.

Treatment of Opiate Withdrawal with Ibudilast

The present invention also relates to novel anti-inflammatory approaches to treating opioid dependence and withdrawal, and specifically the use of ibudilast as an effective therapeutic treatment for morphine withdrawal. The clinical manifestations of morphine withdrawal are thought to result, in part, from glial activation in the central nervous system (Narita et al. (2006) Nature Neuropsychopharmacology 1-13). Ibudilast is an anti-inflammatory drug with the ability to down-regulate glial cell activation. Mizuno et al. (2004) Neuropharmacology 46: 404-411; Suzumura et al. (1999) Brain Res. 837:203-212; Wakita et al. (2003) Brain Res. 992: 53-59. Systemic (e.g., oral) or central (e.g., intrathecal) administration of ibudilast provides a novel approach to attenuate morphine withdrawal, thereby providing an effective treatment for a condition with few good therapeutic options.

Ibudilast acts to suppress inflammation via action on inflammatory cells (e.g., glial cells) resulting in the suppression of both pro-inflammatory mediator and neuroactive mediator release. While ibudilast (administered systemically) has been extensively explored in several other clinical indications, it has not previously been proposed for relief of morphine withdrawal.

A growing body of literature suggests that repetitive morphine treatment may result in glial cell (microglia, astrocytes) activation, and that such activation may contribute to the sequelae of events associated with morphine tolerance and withdrawal.

Several cues activate glia: immune challenges, infection and/or peripheral inflammation, substances released during prolonged neuron-to-neuron transmission (e.g., neurotransmitters, nitric oxide, prostaglandins, substance P, fractalkine, etc.), neuronal damage (e.g., fractalkine, heat shock proteins, cell wall components), etc. Glial function is changed dramatically upon activation, resulting in elevated release of neuroactive substances. Such events are thought to contribute to altered neurological function with manifestations ranging from neurodegeneration, to pain facilitation, to sensitization of morphine dependence and subsequent withdrawal syndrome. Watkins and Maier (2002) Physiol. Rev. 82: 981-1011; Watkins and Maier (2004) Drug Disc. Today: Ther. Strategies 1(1): 83-88, etc.

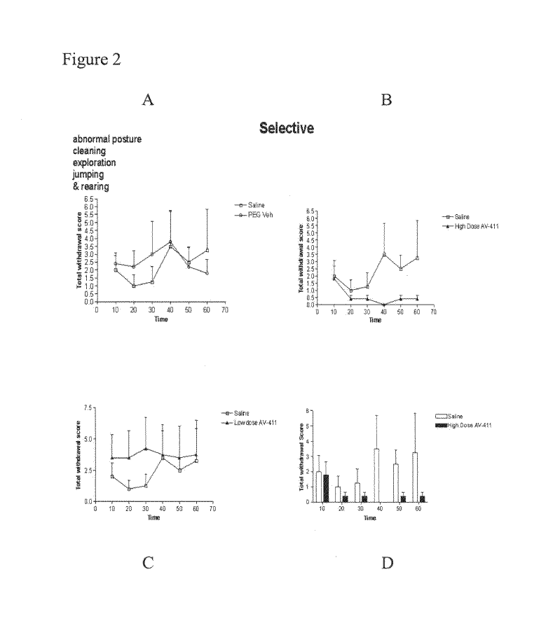

According to the present invention, ibudilast can be used to reduce this undesired glial activation. Ibudilast crosses the blood-brain barrier when administered systemically (Sugiyama et al. (1993) No To Shinkei 45(2):139-42; see also FIG. 2 herein), eliminating the need for more invasive methods of administration in order to access central sites of inflammation involved in pathogenesis of morphine dependence and withdrawal. While certain agents like minocycline and fluorocitrate may have some activity preventing glial activation, they are unacceptable for human therapy. Fluorocitrate is unacceptable because it can block glial uptake of excitatory amino acids (Berg-Johnsen et al. (1993) Exp. Brain Res. 96(2):241-6), an essential function of glia in the maintenance of normal CNS homeostasis, and extended duration or increased doses of fluorocitrate cause seizures. Willoughby J. O., et al. (2003) J. Neurosci. Res. 74(1):160-66; Hornfeldt, C. S. and Larson, A. A. (1990) Eur. J. Pharmacol. 179(3):307-13. While minocycline may be useful in preventing glial activation, it does not appear to be able to reverse extant situations. Raghavendra et al. (2003) J. Pharmacol. and Exp. Therapeutics 306: 624-30; Ledeboer, A., et al. (2005) Pain 115:71-83.

Taken together, glia and their pro-inflammatory or neuromodulatory products may present opportunities for new strategies for control of morphine withdrawal. In one embodiment of the present invention, ibudilast is used to block the release of pro-inflammatory cytokines and neuromodulatory substances. Ibudilast is a potent suppressor of glial activation. Mizuno et al. (2004) Neuropharmacology 46:404-11. In a dose-dependent manner, ibudilast suppressed the production of nitric oxide (NO), reactive oxygen species, interleukin (IL)-1β, IL-6, and tumor necrosis factor (TNF) and enhanced the production of the inhibitory cytokine, IL-10, and additional neurotrophic factors, including nerve growth factor (NGF), glia-derived neurotrophic factor (GDNF), and neurotrophin (NT)-4 in activated microglia.

In one embodiment of the present invention, ibudilast is administered systemically or intrathecally in human subjects for the treatment of morphine withdrawal syndromes.

In other embodiments, ibudilast is administered by systemic (e.g., oral) or central (e.g., intrathecal) routes to attenuate neuropathological elements of morphine withdrawal.

Additional information is available in the following publications, the disclosures of which are hereby incorporated by reference in their entireties: Obernolte, R., et al. (1993) Gene 129:239-47; Rile, G., et al. (2001) Thromb. Res. 102:239-46; Souness, J. E., et al. (1994) Br. J. Pharmacol. 111:1081-88; Suzumura, A., et al. (1999) Brain Res. 837:203-12; Takuma, K., et al. (2001) Br. J. Pharmacol. 133:841-848.

Ibudilast may also be administered in combination with one or more other agents as part of a comprehensive opioid withdrawal treatment protocol. Such agents include, but are not limited to, the following agents:

Naltrexone (17-(cyclopropylmethyl)-4,5α-epoxy-3,14-dihydroxymorphinan-6-one, CAS No. 16676-29-2 (HCl)) has the molecular formula C₂₀H₂₃NO₄ and a molecular weight of 341.4.

Metoclopramide (4-amino-5-chloro-N-(2-diethylaminoethyl)-2-methoxy-benzamide, CAS No. 364-62-5) has the molecular formula C₁₄H₂₂ClN₃O₂ and a molecular weight of 299.8.

Loperamide (4-[4-(4-chlorophenyl)-4-hydroxy-1-piperidyl]-N,N-dimethyl-2,2-diphenyl-butanamide, CAS No. 53179-11-6) has the molecular formula C₂₉H₃₃ClN₂O₂ and a molecular weight of 477.04.

Diazepam (10-chloro-6-methyl-2-phenyl-3,6-diazabicyclo[5.4.0]undeca-2,8,10,12-tetraen-5-one, CAS No. 439-14-5) has the molecular formula C₁₆H₁₃ClN₂O and a molecular weight of 284.74.

Clonidine (2-(2,6-dichlorophenylamino)-2-imidazoline hydrochloride, CAS No. 4205-90-7) has the molecular formula C₉H₉CI₂N_{3—}HCl and a molecular weight of 266.56.

Paracetemol (N-(4-hydroxyphenyl)ethanamide, CAS No. 103-90-2), also referred to as acetaminophen, has the molecular formula C₈H₉NO₂ and a molecular weight of 151.2.

### Pharmaceutical Compositions for Treating Addiction IBUDILAST

Ibudilast is a small molecule drug (molecular weight of 230.3) having the structure shown below.

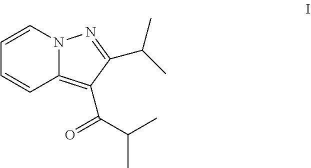

Ibudilast is also found under ChemBank ID 3227, CAS # 50847-11-5, and Beilstein Handbook Reference No. 5-24-03-00396. Its molecular formula corresponds to [C₁₄H₁₈N₂O]. Ibudilast is also known by various chemical names which include 2-methyl-1-(2-(1-methylethyl)pyrazolo(1,5-a)pyridin-3-yl)1-propanone; 3-isobutyryl-2-isopropylpyrazolo(1,5-a)pyridine]; and 1-(2-isopropyl-pyrazolo[1,5-a]pyridin-3-yl)-2-methyl-propan-1-one. Other synonyms for ibudilast include Ibudilastum (Latin), BRN 0656579, KC-404, and the brand name Ketas®. Ibudilast, as referred to herein, is meant to include any and all pharmaceutically acceptable salt forms thereof, prodrug forms (e.g., the corresponding ketal), and the like, as appropriate for use in its intended formulation for administration.

Ibudilast is a non-selective nucleotide phosphodiesterase (PDE) inhibitor (most active against PDE-3, PDE-4, PDE-10, and PDE-11 (Gibson et al. (2006) Eur. J. Pharmacology 538:39-42)), and has also been reported to have LTD4 and PAF antagonistic activities. Its profile appears effectively anti-inflammatory and unique in comparison to other PDE inhibitors and anti-inflammatory agents. PDEs catalyze the hydrolysis of the phosphoester bond on the 3′-carbon to yield the corresponding 5″-nucleotide monophosphate. Thus, they regulate the cellular concentrations of cyclic nucleotides. Since extracellular receptors for many hormones and neurotransmitters utilize cyclic nucleotides as second messengers, the PDEs also regulate cellular responses to these extracellular signals. There are 11 families of PDEs: Ca²⁺/calmodulin-dependent PDEs (PDE1); cGMP-stimulated PDEs (PDE2); cGMP-inhibited PDEs (PDE3); cAMP-specific PDEs (PDE4); cGMP-binding PDEs (PDE5); photoreceptor PDEs (PDE6); high affinity, cAMP-specific PDEs (PDE7); specific PDE (PDE8); high affinity cGMP-specific PDEs (PDE9); and mixed cAMP and cGMP PDEs (PDE10, PDE11).

As stated previously, a reference to any one or more of the herein-described drugs, in particular ibudilast, is meant to encompass, where applicable, any and all enantiomers, mixtures of enantiomers including racemic mixtures, prodrugs, pharmaceutically acceptable salt forms, hydrates (e.g., monohydrates, dihydrates, etc.), different physical forms (e.g., crystalline solids, amorphous solids), metabolites, and the like.

### Formulation Components

Excipients/Carriers

Optionally, in addition to ibudilast, the compositions of the invention may further comprise one or more pharmaceutically acceptable excipients or carriers. Exemplary excipients include, without limitation, carbohydrates, starches (e.g., corn starch), inorganic salts, antimicrobial agents, antioxidants, binders/fillers, surfactants, lubricants (e.g., calcium or magnesium stearate), glidants such as talc, disintegrants, diluents, buffers, acids, bases, film coats, combinations thereof, and the like.

A composition of the invention may include one or more carbohydrates such as a sugar, a derivatized sugar such as an alditol, aldonic acid, an esterified sugar, and/or a sugar polymer. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like.

Also suitable for use in the compositions of the invention are potato and corn-based starches such as sodium starch glycolate and directly compressible modified starch.

Further representative excipients include inorganic salt or buffers such as citric acid, sodium chloride, potassium chloride, sodium sulfate, potassium nitrate, sodium phosphate monobasic, sodium phosphate dibasic, and combinations thereof.

An ibudilast-containing composition of the invention may also include an antimicrobial agent, e.g., for preventing or deterring microbial growth. Non-limiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

A composition of the invention may also contain one or more antioxidants. Antioxidants are used to prevent oxidation, thereby preventing the deterioration of the drug(s) or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

Additional excipients include surfactants such as polysorbates, e.g., “Tween 20” and “Tween 80,” and pluronics such as F68 and F88 (both of which are available from BASF, Mount Olive, N.J.), sorbitan esters, lipids (e.g., phospholipids such as lecithin and other phosphatidylcholines, and phosphatidylethanolamines), fatty acids and fatty esters, steroids such as cholesterol, and chelating agents, such as EDTA, zinc and other such suitable cations.

Further, a composition of the invention may optionally include one or more acids or bases. Non-limiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the role of the excipient, the dosage requirements of the active agent components, and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects.

Generally, however, the excipient will be present in the composition in an amount of about 1% to about 99% by weight, preferably from about 5% to about 98% by weight, more preferably from about 15 to about 95% by weight of the excipient. In general, the amount of excipient present in an ibudilast composition of the invention is selected from the following: at least about 2%, 5%, 10%, 15%, 20%, 25%, 30%, 35%, 40%, 45%, 50%, 55%, 60%, 65%, 70%, 75%, 80%, 85%, 90%, or even 95% by weight.

These foregoing pharmaceutical excipients along with other excipients are described in “Remington: The Science & Practice of Pharmacy”, 19^{th} ed., Williams & Williams, (1995), the “Physician's Desk Reference”, 52^{nd} ed., Medical Economics, Montvale, N.J. (1998), and Kibbe, A. H., Handbook of Pharmaceutical Excipients, 3^{rd} Edition, American Pharmaceutical Association, Washington, D.C., 2000.

Other Actives

A formulation (or kit) in accordance with the invention may contain, in addition to ibudilast, one or more additional active agents effective in treating addiction. Preferably, the active agent is one that possesses a mechanism of action different from that of ibudilast. Such actives include naltrexone, metoclopramide, loperamide, diazepam, clonidine, lofexidine, and paracetemol.

Sustained Delivery Formulations

Preferably, the compositions are formulated in order to improve stability and extend the half-life of ibudilast. For example, ibudilast may be delivered in sustained-release formulations. Controlled or sustained-release formulations are prepared by incorporating ibudilast into a carrier or vehicle such as liposomes, nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures. Additionally, ibudilast can be encapsulated, adsorbed to, or associated with, particulate carriers. Examples of particulate carriers include those derived from polymethyl methacrylate polymers, as well as microparticles derived from poly(lactides) and poly(lactide-co-glycolides), known as PLG. See, e.g., Jeffery et al., Pharm. Res. (1993) 10:362-368; and McGee et al., J. Microencap. (1996).

### Delivery Forms

The ibudilast compositions described herein encompass all types of formulations, and in particular, those that are suited for systemic or intrathecal administration. Oral dosage forms include tablets, lozenges, capsules, syrups, oral suspensions, emulsions, granules, and pellets. Alternative formulations include aerosols, transdermal patches, gels, creams, ointments, suppositories, powders or lyophilates that can be reconstituted, as well as liquids. Examples of suitable diluents for reconstituting solid compositions, e.g., prior to injection, include bacteriostatic water for injection, dextrose 5% in water, phosphate-buffered saline, Ringer's solution, saline, sterile water, deionized water, and combinations thereof. With respect to liquid pharmaceutical compositions, solutions and suspensions are envisioned.

In turning now to oral delivery formulations, tablets can be made by compression or molding, optionally with one or more accessory ingredients or additives. Compressed tablets are prepared, for example, by compressing in a suitable tabletting machine, the active ingredients in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) and/or surface-active or dispersing agent.

Molded tablets are made, for example, by molding in a suitable tabletting machine, a mixture of powdered compounds moistened with an inert liquid diluent. The tablets may optionally be coated or scored, and may be formulated so as to provide slow or controlled release of the active ingredients, using, for example, hydroxypropylmethyl cellulose in varying proportions to provide the desired release profile. Tablets may optionally be provided with a coating, such as a thin film, sugar coating, or an enteric coating to provide release in parts of the gut other than the stomach. Processes, equipment, and toll manufacturers for tablet and capsule making are well-known in the art.

Formulations for topical administration in the mouth include lozenges comprising the active ingredients, generally in a flavored base such as sucrose and acacia or tragacanth and pastilles comprising the active ingredients in an inert base such as gelatin and glycerin or sucrose and acacia.

A pharmaceutical composition for topical administration may also be formulated as an ointment, cream, suspension, lotion, powder, solution, paste, gel, spray, aerosol or oil.

Alternatively, the formulation may be in the form of a patch (e.g., a transdermal patch) or a dressing such as a bandage or adhesive plaster impregnated with active ingredients and optionally one or more excipients or diluents. Topical formulations may additionally include a compound that enhances absorption or penetration of the ingredients through the skin or other affected areas, such as dimethylsulfoxidem bisabolol, oleic acid, isopropyl myristate, and D-limonene, to name a few.

For emulsions, the oily phase is constituted from known ingredients in a known manner. While this phase may comprise merely an emulsifier (otherwise known as an emulgent), it desirably comprises a mixture of at least one emulsifier with a fat and/or an oil. Preferably, a hydrophilic emulsifier is included together with a lipophilic emulsifier that acts as a stabilizer. Together, the emulsifier(s) with or without stabilizer(s) make up the so-called emulsifying wax, and the wax together with the oil and/or fat make up the so-called emulsifying ointment base which forms the oily dispersed phase of cream formulations. Illustrative emulgents and emulsion stabilizers include Tween 60, Span 80, cetostearyl alcohol, myristyl alcohol, glyceryl monostearate and sodium lauryl sulfate. Formulations for rectal administration are typically in the form of a suppository with a suitable base comprising, for example, cocoa butter or a salicylate.

Formulations suitable for vaginal administration generally take the form of a suppository, tampon, cream, gel, paste, foam or spray.

Formulations suitable for nasal administration, wherein the carrier is a solid, include a coarse powder having a particle size, for example, in the range of about 20 to about 500 microns. Such a formulation is typically administered by rapid inhalation through the nasal passage, e.g., from a container of the powder held in proximity to the nose. Alternatively, a formulation for nasal delivery may be in the form of a liquid, e.g., a nasal spray or nasal drops.

Aerosolizable formulations for inhalation may be in dry powder form (e.g., suitable for administration by a dry powder inhaler), or, alternatively, may be in liquid form, e.g., for use in a nebulizer. Nebulizers for delivering an aerosolized solution include the AERx™ (Aradigm), the Ultravent® (Mallinkrodt), and the Acorn II® (Marquest Medical Products). A composition of the invention may also be delivered using a pressurized, metered dose inhaler (MDI), e.g., the Ventolin® metered dose inhaler, containing a solution or suspension of a combination of drugs as described herein in a pharmaceutically inert liquid propellant, e.g., a chlorofluorocarbon or fluorocarbon.

Formulations suitable for parenteral administration include aqueous and non-aqueous isotonic sterile solutions suitable for injection, as well as aqueous and non-aqueous sterile suspensions.

Parenteral formulations of the invention are optionally contained in unit-dose or multi-dose sealed containers, for example, ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the types previously described.

A formulation of the invention may also be a sustained release formulation, such that each of the drug components is released or absorbed slowly over time, when compared to a non-sustained release formulation. Sustained release formulations may employ pro-drug forms of the active agent, delayed-release drug delivery systems such as liposomes or polymer matrices, hydrogels, or covalent attachment of a polymer such as polyethylene glycol to the active agent.

In addition to the ingredients particularly mentioned above, the formulations of the invention may optionally include other agents conventional in the pharmaceutical arts and particular type of formulation being employed, for example, for oral administration forms, the composition for oral administration may also include additional agents as sweeteners, thickeners or flavoring agents.

The compositions of the present invention may also be prepared in a form suitable for veterinary applications.

### Method of Administration

As set forth above, preferred methods of delivery of ibudilast-based therapeutic formulations for the treatment of addictions include systemic and localized delivery, i.e., directly into the central nervous system. Such routes of administration include but are not limited to, oral, intra-arterial, intrathecal, intramuscular, intraperitoneal, subcutaneous, intravenous, intranasal, and inhalation routes.

More particularly, an ibudilast-containing formulation of the present invention may be administered for therapy by any suitable route, including without limitation, oral, rectal, nasal, topical (including transdermal, aerosol, buccal and sublingual), vaginal, parenteral (including subcutaneous, intramuscular, intravenous and intradermal), intrathecal, and pulmonary. The preferred route will, of course, vary with the condition and age of the recipient, the particular neuralgia-associated syndrome being treated, and the specific combination of drugs employed.

One preferred mode of administration for delivery of ibudilast is directly to neural tissue such as peripheral nerves, the retina, dorsal root ganglia, neuromuscular junction, as well as the CNS, e.g., to target spinal cord glial cells by injection into, e.g., the ventricular region, as well as to the striatum (e.g., the caudate nucleus or putamen of the striatum), spinal cord and neuromuscular junction, with a needle, catheter or related device, using neurosurgical techniques known in the art, such as by stereotactic injection (see, e.g., Stein et al., J. Virol. 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

A particularly preferred method for targeting spinal cord glia is by intrathecal delivery, rather than into the cord tissue itself.

Another preferred method for administering the ibudilast-based compositions of the invention is by delivery to dorsal root ganglia (DRG) neurons, e.g., by injection into the epidural space with subsequent diffusion to DRG. For example, an ibudilast-based composition can be delivered via intrathecal cannulation under conditions where ibudilast is diffused to DRG. See, e.g., Chiang et al., Acta Anaesthesiol. Sin. (2000) 38:31-36; Jain, K.K., Expert Opin. Investig. Drugs (2000) 9:2403-2410.

Yet another mode of administration to the CNS uses a convection-enhanced delivery (CED) system. In this way, ibudilast can be delivered to many cells over large areas of the CNS. Any convection-enhanced delivery device may be appropriate for delivery of ibudilast. In a preferred embodiment, the device is an osmotic pump or an infusion pump. Both osmotic and infusion pumps are commercially available from a variety of suppliers, for example Alzet Corporation, Hamilton Corporation, Alza, Inc., Palo Alto, Calif.). Typically, an ibudilast-based composition of the invention is delivered via CED devices as follows. A catheter, cannula or other injection device is inserted into CNS tissue in the chosen subject. Stereotactic maps and positioning devices are available, for example from ASI Instruments, Warren, Mich. Positioning may also be conducted by using anatomical maps obtained by CT and/or MRI imaging to help guide the injection device to the chosen target. For a detailed description regarding CED delivery, see U.S. Pat. No. 6,309,634, incorporated herein by reference in its entirety.

An ibudilast composition of the invention, when comprising more than one active agent, may be administered as a single combination composition comprising a combination of ibudilast and at least one additional active agent effective in the treatment of addiction. In terms of patient compliance and ease of administration, such an approach is preferred, since patients are often adverse to taking multiple pills or dosage forms, often multiple times daily, over the duration of treatment. Alternatively, albeit less preferably, the combination of the invention is administered as separate dosage forms. In instances in which the drugs comprising the therapeutic composition of the invention are administered as separate dosage forms and co-administration is required, ibudilast and each of the additional active agents may be administered simultaneously, sequentially in any order, or separately.

Kits

Also provided herein is a kit containing at least one combination composition of the invention, accompanied by instructions for use.

For example, in instances in which each of the drugs themselves are administered as individual or separate dosage forms, the kit comprises ibudilast in addition to each of the drugs making up the composition of the invention, along with instructions for use. The drug components may be packaged in any manner suitable for administration, so long as the packaging, when considered along with the instructions for administration, clearly indicates the manner in which each of the drug components is to be administered.

For example, for an illustrative kit comprising ibudilast and naltrexone, the kit may be organized by any appropriate time period, such as by day. As an example, for Day 1, a representative kit may comprise unit dosages of each of ibudilast and naltrexone. If each of the drugs is to be administered twice daily, then the kit may contain, corresponding to Day 1, two rows of unit dosage forms of each of ibudilast and naltrexone, along with instructions for the timing of administration. Alternatively, if one or more of the drugs differs in the timing or quantity of unit dosage form to be administered in comparison to the other drug members of the combination, then such would be reflected in the packaging and instructions. Various embodiments according to the above may be readily envisioned, and would of course depend upon the particular combination of drugs, in addition to ibudilast, employed for treatment, their corresponding dosage forms, recommended dosages, intended patient population, and the like. The packaging may be in any form commonly employed for the packaging of pharmaceuticals, and may utilize any of a number of features such as different colors, wrapping, tamper-resistant packaging, blister paks, dessicants, and the like.

Dosages

Therapeutic amounts can be empirically determined and will vary with the particular condition being treated, the subject, and the efficacy and toxicity of each of the active agents contained in the composition. The actual dose to be administered will vary depending upon the age, weight, and general condition of the subject as well as the severity of the condition being treated, the judgment of the health care professional, and particular combination being administered.

Therapeutically effective amounts can be determined by those skilled in the art, and will be adjusted to the requirements of each particular case. Generally, a therapeutically effective amount of ibudilast will range from a total daily dosage, for example in humans, of about 0.1 and 500 mg/day, more preferably, in an amount between 1 and 200 mg/day, 1 and 100 mg/day, 1 and 40 mg/day, or 1 and 20 mg/day. Administration can be one to three times daily for a time course of one day to several days, weeks, months, and even years, and may even be for the life of the patient.

Practically speaking, a unit dose of any given composition of the invention or active agent can be administered in a variety of dosing schedules, depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, every other day, three times weekly, twice weekly, once weekly, twice monthly, once monthly, and so forth.

### III. EXPERIMENTAL

A. Treatment of Addictions with Ibudilast

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Pharmacokinetics and Tissue Distribution of Ibudilast in Rat

Ibudilast pharmacokinetics and distribution into plasma, muscle, brain, and spinal cord were assessed as follows.

### Experimental Procedures

Ibudilast for administration to rats was prepared in 15% ethanol/saline. Drug stability and concentration were validated by HPLC/MS/MS.

Pathogen-free adult male Sprague-Dawley rats (280-350 g; Harlan Labs) were used in all experiments. Rats were housed in temperature (23+/−3° C.) and light (12:12 light: dark; lights on at 0700 hr) controlled rooms with standard rodent chow and water available ad libitum. Behavioral testing was performed during the light cycle.

Rats (n=3/group) were administered 5 mg/kg ibudilast, i.p., and plasma, muscle, brain, and spinal cord were harvested at 5, 15, 60, 180, and 420 minutes post administration. The concentration of ibudilast in tissue samples was determined as follows. A solution of ibudilast (Haorui) at 0.5 mg/ml in DMSO was used as the working reference stock solution. Calibration standards in plasma were prepared by diluting each 0.5 mg/ml stock 1 in 100 into rat plasma to 5000 ng/ml (5 μl+495 μl), and then diluted further to 2.29 ng/ml by 3-fold serial dilution with plasma. Standards were used as low, mid and high QC samples, respectively.

Calibration standards, QC and plasma study samples were prepared for HPLC injection by precipitating 25 μl of plasma with three volumes (75 μl) of ice cold acetonitrile containing 50 ng/ml diphenhydramine and 100 ng/ml dextromethorphan as the internal standards. Tissue study samples were prepared for HPLC injection by adding 1 μl of water per mg of tissue plus three volumes (relative to water) of ice cold acetonitrile containing 50 ng/ml diphenhydramine and 100 ng/ml dextromethorphan as the internal standards, then homogenizing with an electric homogenizer. Following centrifugation at 6100 g for 30 minutes, 40 μl of each supernatant was diluted with 200 μl of 0.2% formic acid in water and analyzed under the following LC/MS/MS conditions:

| | |
|---|---|
| HPLC: | Shimadzu VP System |
| Mobile Phase: | 0.2% formic acid in water (A) and in methanol (B) |
| Column: | 2 × 10 mm Peeke Scientific DuraGel G C₁₈ guard |
| | cartridge |
| Injection Volume: | 100 μl |
| Gradient: | 5-95% B in 2 minutes after a 0.75 minute wash |
| Flow Rate: | 400 μl/min |
| Mass Spectrometer: | Applied Biosystems/MDS SCIEX API 3000 |
| Interface: | TurboIonSpray (ESI) at 400° C. |
| Ionization Mode: | Positive Ion |
| Q1/Q3 Ions: | 231.2/161.2 for Ibudilast (IBUDILAST) |

### Results

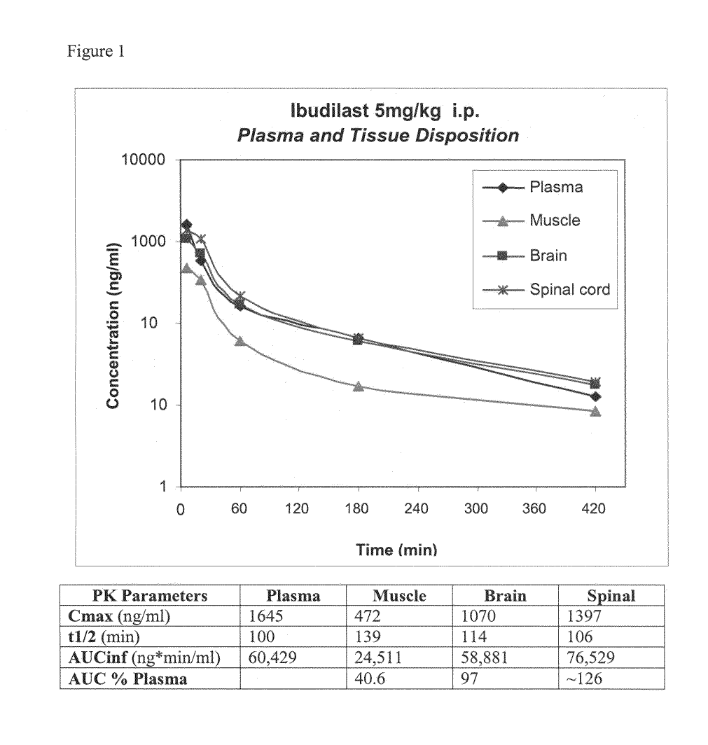

As shown in FIG. 1, intraperitoneal administration of ibudilast yielded good plasma concentrations that declined from Cmax in a biphasic manner. Ibudilast was well distributed to peripheral (e.g., muscle) and central (e.g., brain and spinal cord) tissues. The maximal concentration (Cₘₐₓ) in plasma and CNS tissues was ˜1 μg/mL following i.p. administration of ˜5 mg/kg ibudilast formulated as described. The elimination half-life ranged from 100-139 min in all tissue compartments.

### Example 2

### Efficacy of Ibudilast in a Rat Model of Morphine Withdrawal

A study lasting approximately one week was performed to assess the potential for ibudilast co-treatment to reduce the intensity and duration of morphine withdrawal behaviors.

### Experimental Procedures

Ibudilast was obtained as a pure powder from Sigma (St. Louis, Mo.) or Haorui Pharma (Edison, N.J.) and prepared daily as a solution for intraperitoneal (i.p.) administration. Previous range-finding tolerability and efficacy studies in other neurological models indicated that ibudilast was well-tolerated intraperitoneally at dose levels up to 15 mg/kg twice a day (bid) for multiple days. Ibudilast efficacy following intraperitoneal administration was representative of other systemic routes of administration such as oral treatment. An appropriate amount of ibudilast was dissolved in 100% polyethylene (PEG) 400 (Sigma) and then diluted down to a final concentration of 35% PEG400 in sterile saline (0.9% for injection).

Ibudilast was administered at 2.5 mg/kg (0.9 ml/kg of 2.8 mg/ml in 35% PEG/saline), or 7.5 mg/kg (2.7 ml/kg of 2.8 mg/ml in 35% PEG/saline) each morning (typically 9 am) and afternoon (typically 4 pm). Drug stability and concentration were validated by HPLC/MS/MS.

Pathogen-free adult male Sprague-Dawley rats (280-350 g; Harlan Labs) were used in all experiments. Rats were housed in temperature (23+/−3° C.) and light (12:12 light:dark; lights on at 0700 hr) controlled rooms with standard rodent chow and water available ad libitum. Behavioral testing was performed during the light cycle. Approval of the Institutional Animal Care and Use Committee at University of Colorado was obtained for all procedures.

The schedule for morphine treatment (via subcutaneous injections) was as follows: Day 1: 5 mg/kg at 1000 hr, 5 mg/kg at 1300 hr, 5 mg/kg at 1700 hr; Day 2: 5 mg/kg at 1000 hr, 12.5 mg/kg at 1700 hr; Day 3: 15 mg/kg at 1000 hr; Day 4: 17.5 mg/kg at 1000 hr; Day 5: 5 mg/kg at 1000 hr, 17.5 mg/kg at 1200 hr.

Rats received morphine according to the schedule above, plus either saline (n=4), PEG vehicle (n=5), 2.5 mg/kg (“low dose”) ibudilast (n=4), or 7.5 mg/kg (“high dose”) ibudilast (n=5) according to the following schedule: The two days prior to start of morphine: daily at 1000 hr and 1700 hr; Days 1-4 of morphine regimen: daily at 1000 hr and 1700 hr; Day 5 of morphine regimen: at 1000 hr and 1200 hr. Rats then received 5 mg/kg naloxone at 1245 hr on Day 5, 45 minutes after their last dose of morphine and/or ibudilast, saline or vehicle.

The withdrawal signs measured were: (1) abnormal posturing (an animal presses his abdomen and lower jaw against the floor of the cage); (2) exploration (an animal circles around the cage, thrusting its head in several directions and examining its surroundings); (3) jumping; (4) cleaning (grooming); (5) rearing (an animal stands on its hindpaws with the forepaws off the ground). The total incidence of all five of the stereotyped behaviors in 10 minutes of observation was scored according to the following scale: 0=none displayed; 1=1-5 episodes of a behavior; 2=6-10 episodes of a behavior; 3=11-15 episodes of a behavior; 4=16-20 episodes of a behavior 5=21 or more episodes of a behavior.

Withdrawal scores were measured by blinded observers in 10 minute blocks for 60 minutes immediately after naloxone precipitated withdrawal was initiated. The observations were pooled from 1-10 minutes, 11-20 minutes, 21-30 minutes, 31-40 minutes, 41-50 minutes, 51-60 minutes for each individual rat after naloxone administration, giving six time points. The average score (for each time point) for all animals within an experimental group was reported as the “total withdrawal score” in FIGS. 2A-2D.

### Results

FIGS. 2A-2D demonstrate that ibudilast treatment was effective at reducing both the magnitude and duration of classic physiological manifestations of naloxone-precipitated morphine withdrawal syndrome. While the PEG vehicle had no effect on these behaviors, compared to saline controls (FIG. 2A), ibudilast revealed a dose dependent reduction of these behaviors (FIGS. 2B-2D). Although the low dose of ibudilast (2.5 mg/kg) had no effect compared to saline controls (FIG. 2C), the high dose of ibudilast (7.5 mg/kg) remarkably attenuated behavioral signs of withdrawal (FIG. 2B; presented as a bar graph in FIG. 2D).

### Example 3

### Ibudilast Suppression of Dopamine Release in the Nucleus accumbens

### Experimental Procedures

Dopamine release in the nucleus accumbens is thought to mediate the “reward” associated with drugs of abuse. Ibudilast suppressed dopamine release in the nucleus accumbens, as measured by in vivo microdialysis. Systemic ibudilast (7.5 mg/kg b.i.d.) was co-administered with systemic morphine to rats (6 rats/group) across 5 days, using the morphine regimen described in Example 1. On the morning of the 6^{th} day, rats received ibudilast one hour prior to initiation of baseline sampling. After 3 baseline samples (20 minute inter-sample interval), morphine was administered to all rats. Dialysis samples were collected at 20 minute intervals for 180 minutes. To test behavioral withdrawal and reversal of morphine-induced dopamine, all rats were administered the opioid antagonist naloxone after the 60 minute sample time was completed.

### Results

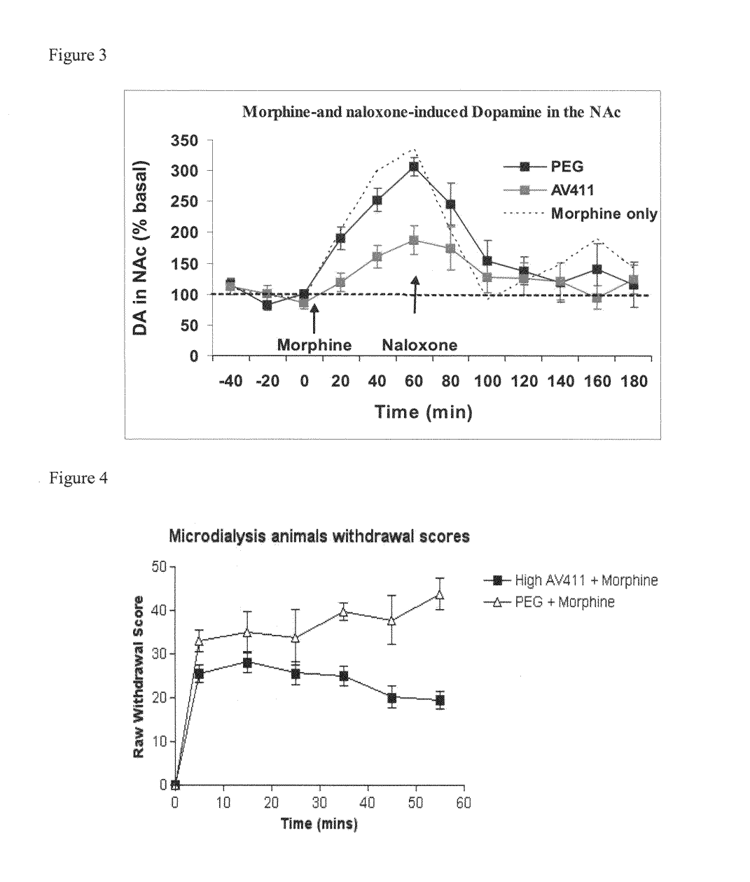

As shown in FIG. 3, rats treated with ibudilast exhibited significantly suppressed indicators or mediators of “reward” as evidenced by suppressed release of dopamine into the nucleus accumbens in response to morphine. Ibudilast did not decrease basal levels of dopamine. The opioid antagonist naloxone reversed the morphine-induced dopamine release, which shows that the dopamine release was indeed due to the effects of morphine. Rats repetitively co-administered ibudilast and morphine showed suppressed naloxone-induced behavioral withdrawal signs, compared to rats repetitively administered PEG-saline vehicle and morphine (see FIG. 4).

### Conclusion

The results of both brain microdialysis dopamine levels and concomitant opiate withdrawal behavioral responses indicate that ibudilast treatment of rats significantly reduces a neurochemical mediator (dopamine) of reward or salience and behavioral manifestations of opiate dependence. Such results imply that ibudilast will be useful for the treatment of multiple forms of dependence.

### Example 4

### Ibudilast Reduces the Development of Morphine Dependence and Central Glial Cell Activation

### Experimental Procedures

Rats (n=10/group) received morphine according to the schedule described above in Example 2, plus either saline, PEG vehicle, 2.5 mg/kg (“low dose”) ibudilast, or 7.5 mg/kg (“high dose”) ibudilast according to the following schedule: The two days prior to start of morphine: daily at 1000 hr and 1700 hr; Days 1-4 of morphine regimen: daily at 1000 hr and 1700 hr; Day 5 of morphine regimen: at 1000 hr and 1200 hr. Rats then received 5 mg/kg naloxone at 1245 hr on Day 5, 45 minutes after their last dose of morphine and/or ibudilast, saline or vehicle.

Following scoring of withdrawal behaviors animals received an intraperitoneal injection of 0.8 ml of 50 mg/ml sodium pentobarbital, and once anesthetized, animals were transcardially perfused. Half of each treatment group were perfused with saline and half with 4% paraformaldehyde. Spinal cord and brains were collected (saline perfusion for protein and mRNA quantification and paraformaldehyde perfusion for immunohistochemistry). Samples collected from saline perfused animals were flash frozen in liquid nitrogen and stored at −80° C. Paraformaldehyde perfused samples were stored in 4% paraformaldehyde for 48 hours and then transferred to 30% sucrose (0.1% azide) until tissue sectioning.

Immunoreactivity for OX-42 (antibody that recognizes complement type 3 receptors, e.g., CD11b) and/or glial fibrillary acidic protein (GFAP), microglial and astrocyte activation markers, respectively, were assessed. Sections (20 μm) were treated with 0.3% H₂O₂ in Tris-buffered saline (TBS) for 20 minutes at room temperature to suppress endogenous peroxidase activity. Sections were then incubated overnight at 4° C. in monoclonal mouse anti-rat OX-42 (1:100; Pharmingen, San Diego, Calif.) or monoclonal mouse anti-rat GFAP antibody (1:200; Chemicon, Temecula, Calif.) in TBS with 2% normal goat serum and 0.5% Triton-X-100. Subsequently, sections were incubated with the appropriate secondary biotinylated antibodies (1:400; Jackson ImmunoResearch, West Grove, Pa.) for 2 hours at room temperature, incubated in avidin-biotin complex solution (ABC; 1:200; Vector Laboratories, Burlingame, Calif.) for 2 hours at room temperature, followed by reaction with 0.5 mg/ml 3,3′-diaminobenzidine tetrahydrochloride (DAB; Sigma). Finally, sections were dried, dehydrated, and coverslipped with Permount. Staining was evaluated by light microscopy. Densitometry of immunohistochemical staining was subsequently evaluated using computer software (NIH image).

Amplification of cDNA was performed using the QUANTITECT SYBR GREEN PCR kit (Qiagen, Valencia, Calif.) in ICYCLER IQ 96-well PCR plates (Bio-Rad, Hercules, Calif.) on a MYIQ single color real-time PCR detection system (Bio-Rad). The reaction mixture (26 μl) was composed of 1× QUANTITECT SYBR GREEN PCR master mix (containing the fluorescent dye SYBR green 1,2.5 mM MgCl₂, dNTP mix, and HOTSTART Taq DNA polymerase), 10 nM fluorescein, 500 nM each of forward and reverse primers, 25 ng cDNA and nuclease-free H₂O. Reactions were done in triplicate (n=3-6 animals/group). The reaction conditions were an initial 15 minutes at 95° C., followed by 40 cycles of 15 seconds at 94° C., 30 seconds at 55-60° C., and 30 seconds at 72° C. Melt curve analyses were conducted to assess uniformity of product formation, primer-dimer formation, and amplification of non-specific products. Linearity and efficiency of PCR amplification were assessed using standard curves generated by increasing amounts of cDNA. SYBR green 1 fluorescence (PCR product formation) was monitored in real time using the MYIQ single color real-time PCR detection system (Bio-Rad). Threshold for detection of PCR product was set in the log-linear phase of amplification and the threshold cycle (CT, the number of cycles to reach threshold of detection) was determined for each reaction. The levels of the target mRNAs were quantified relatively to the level of the housekeeping gene glyceraldehyde-3-phosphate-dehydrogenase (GAPDH) using the comparative CT (ACT) method (Livak and Schmittgen, 2001). Expression of the housekeeping gene was not significantly altered by experimental treatment.

### Results

### A. Weight Changes

Animals treated with ibudilast showed reduced body weight loss compared to animals treated with vehicle during the first 2 days of treatment (9.3±6.3 g for animals treated with 7.5 mg/kg ibudilast; 10.4±5.6 g for animals treated with 2.5 mg/kg ibudilast; and 1±4.4 g for animals treated with vehicle). Therefore, data were normalized for weights on the morning animals started morphine treatment (thereby removing the ibudilast induced weight loss during the first 2 days). On day 7, the morphine induced weight loss was 13.3±7.1 g in animals treated with 7.5 mg/kg ibudilast, 16.6±5.7 g in animals treated with 2.5 mg/kg ibudilast, and 18.2±6.6 g in animals treated with vehicle. Body weight loss is a classic and objective marker of withdrawal in rat opiate models and attenuation by high dose ibudilast is supportive of physiological benefit during opiate withdrawal.

### B. Withdrawal Behaviors

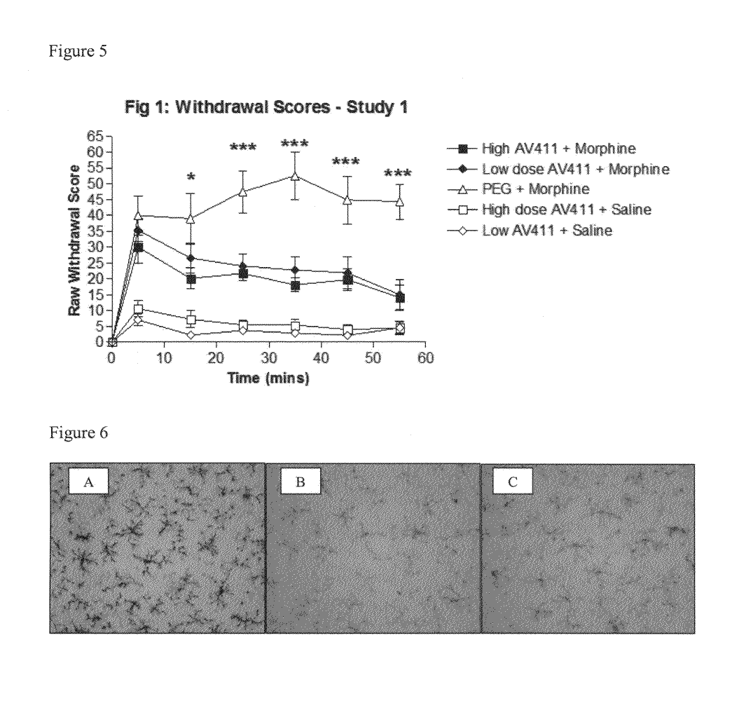

As shown in FIG. 5, treatment with ibudilast at dosages of 2.5 mg/kg and 7.5 mg/kg resulted in a dramatic reduction of naloxone precipitated withdrawal behaviors during a 60 minute observation period. On an individual behavior basis, treatment with ibudilast resulted in reductions in all behaviors except for rearing, exploration and wet dog shakes, whereas no change was observed in animals treated with vehicle. Data are presented in FIG. 5 as the sum of the total withdrawal behaviors observed during each ten minute block for all animals in the study (n=10/treatment group).

### C. Brain Immunohistochemistry

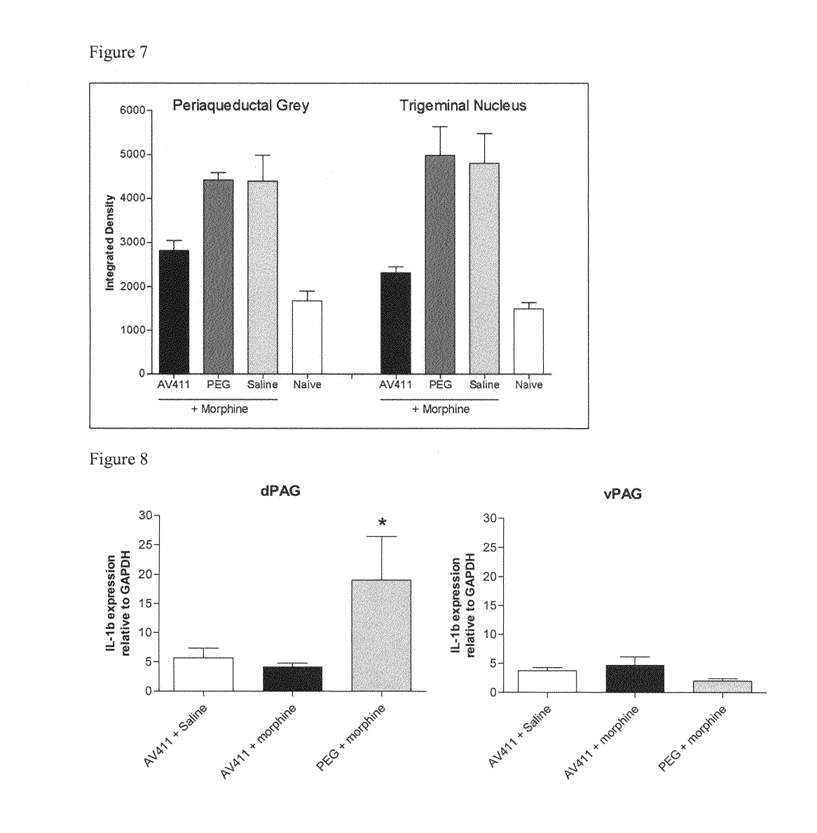

Immunohistochemical analysis was conducted on brain samples collected from rats following paraformaldehyde perfusion. Microglial activation marker CD11b and astrocyte marker GFAP were investigated. As can be seen in FIG. 6, chronic morphine administration caused visible upregulation of the microglia activation marker CD11b. Treatment with ibudilast dramatically reduced the increase in the CD11b marker. Densitometry analysis (FIG. 7) revealed that ibudilast caused a significant reduction in the microglial activation marker CD11b in 2 brain regions, the periaqueductal grey and the brain homologue of the spinal dorsal horn, the trigeminal nucleus.

D. mRNA Analysis of Brain Nuclei

Interleukin-1 mRNA from the brain tissue samples was quantitated. Morphine caused a dramatic increase in interleukin-1 mRNA in the dorsal, but not the ventral periaqueductal grey region (FIG. 8). Ibudilast completely blocked the morphine induced increase in interleukin-1 mRNA in the dorsal periaqueductal grey region.

### Conclusion

Ibudilast administration during morphine treatment results in significantly reduced glial cell activation and proinflammatory cytokine production in the brain of treated animals. Upon naxolone-precipitated withdrawal, animals receiving ibudilast display significantly reduced behavioral responses indicating that ibudilast treatment attenuates the neuroinflammation and behavioral symptoms associated with the syndrome of opiate withdrawal.

### Example 5

### Ibudilast Reverses Morphine Dependence and Spontaneous Opioid Withdrawal

### Experimental Procedures

Pathogen-free adult male Sprague-Dawley rats were used in all experiments. Rats (350-375 g at the time of arrival; Harlan Labs, Madison, Wis.) were housed in temperature (23±3° C.) and light (12:12 light:dark; lights on at 0700 hours) controlled rooms with standard rodent chow and water available ad libitum. All procedures were approved by the Institutional Animal Care and Use Committee at the University of Colorado at Boulder. Upon arrival male Sprague Dawley rats (300-400 g) were housed individually and allowed to acclimatize to the animal colony telemetry room for one week. 5×90 minute sessions of handling by investigators were performed during the following week.

Rats were anesthetized with isoflurane, and emitters for measuring core body temperature (MiniMitter, Sun River, Oreg.) were implanted in the peritoneal cavity. Gross motor movement was assessed by telemetry using the same emitters used for recording core body temperature. The emitter had to move for activity to be counted; thus, stationary movements such as grooming were not counted. Activity counts and core body temperature were measured every minute and movement averaged over 120 minutes was calculated (thereby smoothing the data). Recording of telemetry data occurred throughout the entire experiment. Periods of time when experimenters entered the housing room were eliminated from analyses as these produced increased activity and therefore error. At the time of telemetry implant, animals were implanted with 2 subcutaneous 2ML2 lumbar osmotic minipumps (Alzet, Cupertino, Calif.), which each pumped at about 5 μl per hour for 14 days (hence a combined total of 10 μl per hour). One pump had a lead length of PE60 tubing pre-loaded with saline to delay the morphine delivery for 2 days. Therefore, the pumps delivered 6.25 mg of morphine (or saline) per day on days 1 and 2, then 12.5 mg per day from then onward. On day 12, animals began a 7 day twice daily ibudilast regimen (7.5 mg/kg or 2.5 mg/kg in 35% PEG in saline dose volume 2.5 ml/kg) or vehicle (35% PEG in saline) (completing with the final dose on the afternoon of day 18). The morning injection occurred between 8:45 AM and 9:15 AM, with the afternoon injection occurring between 4:45 PM and 5:15 PM. On day 14, the pumps were removed to precipitate spontaneous opioid withdrawal (in animals receiving morphine). Body weights were recorded prior to each dosing session to allow for accurate dose calculations and to track the opioid induced weight loss (also occurred on days when no dosing was conducted).

### Results

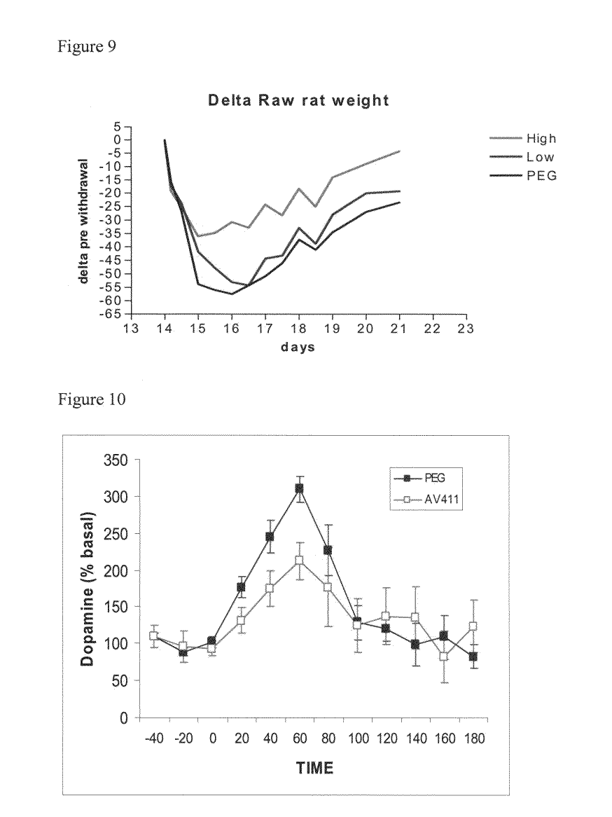

Ibudilast protected animals from spontaneous opioid withdrawal induced weight loss. As shown in FIG. 9, only a trend towards improved weight change was observed at the lower dose of ibudilast, but at the higher dose, ibudilast substantially attenuated weight loss. The endpoint of weight loss attenuation is considered an important objective measurement of reduced withdrawal in rats.

### Example 6

### Nucleus Accumbens Dopamine Microdialysis Following 5 Days of Treatment with Morphine and Ibudilast

### Experimental Procedures

Pathogen-free adult male Sprague-Dawley rats were used in all experiments. Rats (300-325 g at the time of arrival; Harlan Labs, Madison, Wis.) were housed in temperature (23±3° C.) and light (12:12 light:dark; lights on at 0700 hours) controlled rooms with standard rodent chow and water available ad libitum. All procedures were approved by the Institutional Animal Care and Use Committee at the University of Colorado at Boulder. Upon arrival male Sprague Dawley rats (300-400 g) were housed in pairs and allowed to acclimatize to the animal colony for one week. 5×90 minute sessions of handling by investigators and animal acclimatization to the microdialysis environment were performed during the following week.

Microdialysis guide cannula implantation was performed under halothane anaesthesia. CMA 12 guide cannulae (CMA Microdialysis) were aimed at either the right or left nucleus accumbens (AP=+1.7, LM=±0.8, DV=−6.0) in a counterbalanced fashion. Coordinates were from bregma using the atlas of Paxinos and Watson (1998). The guide cannulae and a tether screw (CMA Microdialysis) were anchored to the skull with three jeweler's screws and dental cement. Rats were individually housed after surgery and allowed to recover for one week.

Animals then began a 7 day dosing regimen (groups of 6 animals at a time, n=10 per treatment group). Throughout the 7 days animals received twice daily intraperitoneal injections of ibudilast (7.5 mg/kg or 2.5 mg/kg in 35% PEG in saline dose volume 2.5 ml/kg) or vehicle (35% PEG in saline). The morning injection occurred between 8:45 AM and 9:15 AM, with the afternoon injection occurring between 4:45 PM and 5:15 PM. On day 3 animals began a 5 day dependence regimen of morphine or vehicle (saline) (subcutaneous injections 1 ml/kg). When morphine was administered in the morning or afternoon, it occurred 30 minutes following the ibudilast injection. The dependence regimen consisted of Day 3: AM dose 5 mg/kg, noon dose 5 mg/kg, PM dose 5 mg/kg; Day 4: AM dose 7.5 mg/kg, PM dose 12.5 mg/kg; Day 5 AM dose 15 mg/kg; Day 6 AM dose 17.5 mg/kg; and Day 7 AM dose 22.5 mg/kg. Body weights were recorded prior to each dosing session to allow for accurate dose calculations and to track the opioid induced weight loss.

On the afternoon before microdialysis (day 6 following morphine and ibudilast administration) rats were transferred to the dialysis room that was on the same light-dark cycle as the colony room. Microdialysis probes (CMA 12, MW cut-off 20,000 Da, 2 mm active membrane) were inserted into the guide cannulae and rats were placed in separate Plexiglas infusion bowls with food and water available ad libitum. Ringers solution (147 mM NaCl, 2.97 mM CaC1, 4.02 mM KCl; Baxter) was perfused through the probes using a CMA infusion pump at a flow rate of 0.2 μl/min overnight. The flow rate was increased to 1.5 μl/min the next morning and, after a 1 hour equilibration period, the final dose of morphine was administered and sample collection began and dialysates were collected manually every 20 minutes and immediately placed in −80° C. until analysis. In one set of animals opioid withdrawal was precipitated with 10 mg/kg subcutaneous (dose volume 1 ml/mg) naloxone 60 minutes after the morphine administration. Collection tubes were pre-filled with 3 μl of 0.02% EDTA (anti-oxidant) in 1% ethanol. After collection of three baseline samples, morphine or vehicle was administered in the same manner as described above. Dialysates were analyzed by HPLC within 2 weeks of collection.

Dopamine in the dialysates was determined using an ESA 5600A COULARRAY detector with an ESA 5014B analytical cell and an ESA 5020 guard cell connected to an ESA HR80 column (C18, 3 μm, 80×3 mm) which was maintained at 30° C. The mobile phase was 150 mM sodium dihydrogen phosphate monohydrate, 4.76 mM citric acid monohydrate, 3 mM sodium dodecyl sulfate, 50 μM EDTA, 10% methanol, and 15% acetonitrile, pH=5.6 with sodium hydroxide. The potentials were set at −75 and +220 mV, and the guard cell potential was set at +250 mV. Injections were performed with an ESA 542 autosampler using an injection volume of 27 μl. Quantitative comparisons were made with external standards (Sigma-Aldrich, St Louis, Mo.) that were run each day.

To verify probe placement, rats were euthanized with 65 mg/kg ip sodium pentobarbital. The brains were removed, frozen in chilled isopentane, and cryostat sectioned (40 μm) at −20° C. Sections were mounted on gelatin-treated slides, stained with cresyl violet, and coverslipped. Only rats with probes placed within the nucleus accumbens were included in the analysis.

### Results

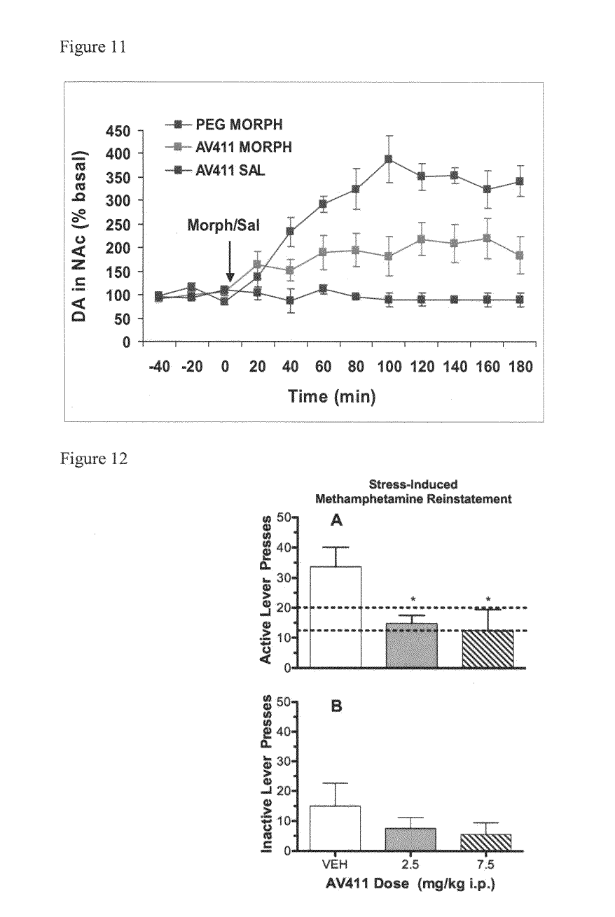

Treatment with ibudilast resulted in dramatically reduced morphine induced nucleus accumbens dopamine increases in morphine-dependent animals during morphine administration and during naloxone precipitated opioid withdrawal or spontaneous opioid withdrawal (FIGS. 10 and 11). FIG. 10 shows ibudilast reduced nucleus accumbens dopamine levels in morphine-dependent animals during naloxone precipitated opioid withdrawal (10 mg/kg of naloxone was administered subcutaneously for 60 minutes) in animals treated with 7.5 mg/kg ibudilast. FIG. 11 shows that ibudilast also reduced nucleus accumbens dopamine levels in morphine-dependent animals following morphine administration (at time 0) during spontaneous opioid withdrawal in animals treated with ibudilast (7.5 mg/kg) or a combination of ibudilast and morphine.

### Conclusions

Ibudilast treatment was shown to significantly reduce the increased dopamine levels observed in the brain nucleus accumbens following morphine treatment in a rat model of morphine dependence. Since drugs of abuse cause increased dopamine in the nucleus accumbens (and this increase is what is thought to mediate the “reward” associated with such drugs), the results imply that ibudilast therapy may similarly reduce dependence and attenuate withdrawal for any addictive disorder. Hence, ibudilast treatment is indicated for not only syndromes associated with opiates, but also for other classes of drugs, such as psychostimulants (cocaine, amphetamine, methamphetamine), cannabinoids, and alcohol. Furthermore, ibudilast treatment could also be extended to potentially attenuate “behavioral addictions” such as gambling and over-eating.

### Stress-Induced or Prime-Induced Methamphetamine Relapse or Reinstatement

Stress and/or association with environmental cues associated with drug “highs” and/or renewed contact with a drug (a “slip”) have been linked to persisting relapse of methamphetamine abuse. Methamphetamine can activate glia in vitro and human brain microglial activation has been linked with methamphetamine abuse. Several studies have indicated that, in vitro treatment with methamphetamine causes long-lasting astrocytic activation in limbic neuron/glia co-cultures (Suzuki et al. 2007) and its in vivo administration has been reported to activate microglia in the striatum of rats and mice (Fantegrossi et al. 2004; LaVoie et al. 2004; Thomas et al. 2004b). Importantly, recent PET imaging of human methamphetamine addicts with a marker of microglial activation has shown upregulated glial activation in methamphetamine abuser which also inversely correlated with duration of abstinence.

In one embodiment, the present invention provides a method for treating or attenuating relapse or reinstatement from a psychostimulant in a subject by administering a PDE inhibitor or a glial attenuator. While the inventive methodlogy is described with respect to prevention of methamphetamine relapse and/or reinstatement, it is stated that the present invention should not be limited to this embodiment. Rather, relapse or reinstatement to any psychostimulant can be prevented, inhibited or treated using the inventive method.

According to one embodiment, the present invention provides a method for inhibiting and/or preventing methamphetamine relapse by administering the glial cell attenuator, 3-isobutyryl-2-isopropylpyrazolo-[1,5-a]pyridine (AV411, ibudilast), a non-selective PDE inhibitor. Specifically, the present inventors studied whether ibudilast could attenuate, inhibit or prevent methamphetamine prime- and stress-induced reinstatement of extinguished response in rats previously reinforced to self-administer methamphetamine. The “reinstatement” procedure was used as a potential predictor of methamphetamine relapse, based on its wide spread acceptance as a preclinical procedure for evaluating potential medications for treating drug abuse relapse. The inventive method is also suitable for preventing or inhibiting relapse or reinstatement to other psychostimulating drugs, such as a drug selected from the group consisting of an amphetamine, a methylenedioxymethamphetamine, a methamphetamine, and a dextroamphetamine.

According to another embodiment, the present invention provides a method for suppressing dopamine release in the nucleus accumbens of a subject suffering from a psychostimulant addiction or dependence by administering ibudilast to such a subject. In one embodiment the inventive method is directed to treating a psychostimulant addiction or dependence to a drug selected from the group consisting of an amphetamine, a methamphetamine, a methylenedioxymethamphetamine, and a dextroamphetamine.

### Example 7

### Treatment of Methamphetamine Reinstatement Using Ibudilast

### A. Experimental Set Up

### Subjects

Adult male Long-Evans hooded rats (Harlan, Indianapolis, Ind.) weighing 275-300 g at the start of studies were acclimated to the vivarium for at least one week prior to catheter implantation. When not in testing, rats were individually housed in standard plastic rodent cages in a temperature-controlled (22° C.), in an American Association of Animal Laboratory Care-accredited facility in which they had ad libitum access to water. The rats were allowed ad libitum rat chow for at least one week prior to commencement of training, after which they were maintained at 320 g by controlled feedings. The rats were maintained on a reversed, 12 hr/12 hr light-dark cycle (0600-1800 lights off) for the duration of the experiment and they were trained and tested during the dark segment of this cycle. Studies were approved by the Institutional Animal Care and Use Committee of the Virginia Commonwealth University and conformed with NIH Guidelines for Care and Use of Laboratory Animals.

### Infusion Assembly System

Catheters were constructed from polyurethane tubing (Access Technologies, Skokie, Ill.; 0.044″ outer diameter×0.025″ inner diameter). The proximal 3.2 cm of the catheter was tapered by stretching following immersion in hot sesame oil. The catheters were prepared with a retaining cuff approximately 3 cm from the proximal end of the catheter. A second larger retaining cuff was positioned approximately 3.4 cm from the proximal end of the catheter. Mid-scapula cannula/connectors were obtained from Plastics One (Roanoke, Va.). The cannula/connectors consisted of a threaded plastic post through which passed an “L” shaped section of 22 gauge stainless steel needle tubing. The lower surface of the plastic post was affixed to a 2 cm diameter disc of Dacron mesh. During sessions the exposed threaded portion of the infusion cannula was connected to an infusion tether consisting of a 35 cm length of 0.40 mm inner diameter polypropylene tubing encased within a 30 cm stainless steel spring to prevent damage. The upper portion of the 0.40 polypropylene tubing was connected to a fluid swivel (Lomir Biomedical, Inc, Quebec, Canada) that was, in turn, attached via 0.40 polypropylene tubing to the infusion syringe.

### Surgical Procedure

Following acclimation to the laboratory environment, indwelling venous catheters were implanted into the right external jugular vein. Surgical anesthesia was induced with a combination of 50 mg/kg ketamine (KetaThesia, Butler Animal Health Supply, Dublin, Ohio) and 8.7 mg/kg xylazine (X-Ject E, Butler Animal Health Supply, Dublin, Ohio). Rats were additionally administered 8 mg/kg oral enrofloxacin (Baytril, Bio-Serv, Frenchtown, N.J.) for three days post-surgery. The ventral neck area and back of the rat were shaved and wiped with povidone-iodine, 7.5% (Betadine, Purdue Products L.P., Stamford, Conn.) and isopropyl alcohol. The rat was placed ventral side down on the surgical table and a 3 cm incision was made 1 cm lateral from mid-scapula. A second 0.5 cm incision was then made mid-scapula. The rat was then placed dorsal side down on the operating table and a 2.5 cm incision was made longitudinally through the skin above the jugular area. The underlying fascia was bluntly dissected and the right external jugular vein isolated and ligated. A small cut was made into the vein using an iris scissors and the catheter was introduced into the vein and inserted up to the level of the larger retaining cuff. The vein encircling the catheter between the two cuffs was then tied with silk suture. A second suture was then used to anchor the catheter to surrounding fascia. The distal end of the catheter was passed subcutaneously and attached to the cannula/connector that was then inserted subcutaneously through the larger incision. The upper post portion of the connector/cannula exited through the smaller mid-scapula incision. All incisions were then sprayed with a gentamicin sulfate/betamethasone valerate topical antibiotic (Betagen, Med-Pharmex, Inc., Pomona, Calif.) and the incisions were closed with Michel wound clips.

Rats were allowed to recover from surgery for at least 5 days before self-administration training began. Periodically throughout training, methohexital (1.5 mg/kg) or ketamine (5 mg/kg) (KetaThesia, Butler Animal Health Supply, Dublin, Ohio) was infused through the catheters to determine patency as inferred when immediate anesthesia was induced. Between sessions the catheters were flushed and filled with 0.1 ml of a 25% glycerol (Acros, N.J.)/75% sterile saline locking solution containing: 250 units/ml heparin (Abraxis Pharmaceutical Products, Schaumburg, Ill.) and 250 mg/ml ticarcillin/9 mg/ml clavulanic acid (Timentin, GlaxoSmithKline, Research Triangle Park, N.C.). If during the experiment a catheter was determined to be in-patent, the left external jugular was then catheterized and the rat was returned to testing. During extinction and reinstatement testing, infusions through catheters did not occur, and these catheter maintenance procedures were not employed.

Briefly, commercially-obtained test chambers equipped with two retractable levers, a 5-w house light, and a Sonalert® tone generator (MED Associates, Inc., St. Albans, Vt.) were used. Positioned above each lever was a white cue light. The grid floors of the chambers were connected to a shock-generating device that was able to deliver 0.63 mA-scrambled foot-shock. A syringe pump (Model PHS-100; MED Associates, Inc., St. Albans, Vt.) when activated, delivered a 6-sec, 0.2 ml infusion. Recording of lever presses and activation of lights, shockers, pumps, and Sonalerts were accomplished by a microcomputer, interface, and associated software (MED-PC® IV, MED Associates, Inc., St. Albans, Vt.).

1. Self-Administration and Extinction Procedures

Methamphetamine self-administration training sessions were conducted five days per week (M-F) for 2 h daily. Each response (fixed ratio 1, FR1) on the right-side lever resulted in delivery of a 0.1 mg/kg methamphetamine infusion (0.2 ml/6 sec) followed by a 14-s timeout period. At the start of an infusion the house light was extinguished, the Sonalert® was sounded, and the cue lights above each lever flashed at 3 Hz. The Sonalert® and cue lights remained activated during the 6 s infusion. Twenty seconds following the onset of the infusion the house light was re-illuminated, and the opportunity to self-administer methamphetamine was again made available (i.e., each methamphetamine infusion initiated a 20 s period during which lever presses were recorded but were without scheduled consequences and further infusions could not be obtained). Active (right-side) lever presses during the infusions as well as all inactive (left-side) lever presses were recorded but were without scheduled consequences.

Self-administration training continued until three criteria had been met: 1) at least 12 self-administration sessions had occurred; 2) at least 15 methamphetamine infusions had occurred during each of the last four sessions; and, 3) at least 125 lifetime methamphetamine infusions had been obtained, after which extinction training began. Subsequently, twelve, two-hour daily (Mon-Sun) extinction sessions were conducted. During extinction sessions, methamphetamine infusions were not delivered. Other conditions during extinction were identical to those during self-administration. That is, during extinction sessions both levers were extended, the houselight was activated, and Sonalert and cue lamp activations occurred as a result of responding according to FR1 schedules.

During the two days immediately prior to the reinstatement testday, AV411 or its vehicle was administered. Multiple administrations of AV411 were given because a minimum period of time (2.5 d) was perceived for it to obtain steady state drug levels in various tissue compartments and to enable minimally sufficient glial attenuation which, in other preclinical procedures had correlated with the onset of efficacy (Hutchinson et al., 2009; Ledeboer et al., 2007; Ledeboer et al., 2006). On the day immediately preceding the reinstatement testday, active lever presses were non-significantly lower in AV411 treated prime and footshock groups, relative to respective vehicle groups. It is important to note that AV411 plasma exposures associated with the dose regimens utilized in these rat studies were at or below those recently reported in clinical trials wherein AV411 was well-tolerated without sedating or related CNS side effects (Johnson et al., unpublished data and Rolan et al., 2008). Overall, the nature of the results lend support to a specific pharmacological action of AV411 to attenuate footshock- or prime-induced methamphetamine relapse, although the exact mechanism(s) are unknown at present.

Thus, for rats scheduled for stress-reinstatement testing, each extinction session was also preceded by a 15-minute period in the operant chamber during which levers were retracted and the house light was not illuminated to parallel the 15-min footshock period during the reinstatement test sessions. For rats scheduled for prime-reinstatement testing, an injection of saline (the vehicle for methamphetamine prime) was administered i.p. 30 min pre-session before the last four or more extinction sessions to habituate the rats to eventual prime injections. AV411 or its vehicle was administered on the last two days of extinction, 60 min pre-session and again at approximately 1600 hrs depending upon the eventual test condition for a rat. Rats were considered to be eligible for reinstatement testing provided that the mean number of active-lever presses during the last 3 sessions of extinction was lower than the mean number of active-lever presses during the first 3 sessions of extinction. Rats that did not meet this extinction criterion were excluded from subsequent testing.

2. Testing the Effectiveness of AV411 in Preventing Footshock-Induced Reinstatement

Reinstatement testing followed extinction training. Conditions during reinstatement testing were identical to those during extinction except that 15 min of intermittent footshock (administered at 0.63 mA, with a 0.5 s activation time and an average inter-activation interval of 40 s) was administered immediately prior to the start of the test session. Rats were administered a dose of AV411 or its vehicle i.p. 60 min prior to the start of the two-hour test session (45 min prior to the start of footshock administration). Different groups of twelve rats each were assigned to each of three reinstatement test conditions: 1) footshock+AV411 vehicle; 2) footshock+2.5 mg/kg AV411; and 3) footshock+7.5 mg/kg AV411.

3. Testing the Effectiveness of AV411 in Preventing Prime-Induced Reinstatement

Reinstatement testing followed extinction training. Conditions during reinstatement testing were identical to those during self-administration conditions except that 1 mg/kg i.p. methamphetamine was administered 30 min pre-session (i.e., methamphetamine prime), AV411 test doses or vehicle were administered 60 min pre-session, and methamphetamine self-administered infusions did not occur. Doses of 0 (vehicle), 2.5 and 7.5 mg/kg i.p. of AV411 were tested using separate groups of 12 rats each.

4. Drugs

(+)-Methamphetamine hydrochloride (#M8750; Sigma-Aldrich, Inc., St. Louis, Mo.) was prepared in sterile 0.9% saline. Methamphetamine stock solutions were sterilized by filtration through 0.2 μm filtration disks. Methamphetamine infusions were delivered in a 6-sec, 0.2 ml volume. Heparin (5 units/ml) was additionally added to methamphetamine and saline infusates. AV411 was supplied by NIDA and was dissolved in a 35% PEG400, 10% Cremophor RH40 aqueous vehicle. AV411 was administered i.p. in 1 ml/kg body weight volume.

5. Data Analysis

Initially, reinstatement testday data were analyzed using the Grubbs test for outliers (Extreme Studentized Deviate) and a rat's data were excluded from subsequent analyses if p<0.05 for its results (GraphPad QuickCalcs Web site: http://www.graphpad.com/quickcalcs/Grubbsl.cfm, accessed April 2008). Numbers of active-lever presses (i.e., the right-side lever, the presses of which were previously-reinforced with methamphetamine) in the vehicle-treated group were compared to those of each AV411 dosage group using Dunnett's one-tailed post-tests (Prism 5 for Macintosh, GraphPad Software, Inc., San Diego, Calif.). This analytical approach was used because the main experimental question was whether treatment with any of the AV411 doses reduced levels of reinstatement. Additionally, the numbers of active-lever presses occurring during the last session of self-administration and during the last-session of extinction amongst groups within the prime and stress reinstatement conditions were compared using an ANOVA (Prism 5 for Macintosh, GraphPad Software, Inc., San Diego, Calif.). If results with the ANOVA were found significant (p<0.05), comparisons between all groups were conducted using Tukey-Kramer tests (Prism 5 for Macintosh, GraphPad Software, Inc., San Diego, Calif.). This analytical approach was used because the experimental questions were whether the groups had been trained to self-administer methamphetamine and to extinguish responding to comparable levels prior to reinstatement testing. A paired, one-tailed t-test was conducted comparing levels of active-lever presses during the last extinction session with those during the reinstatement test session of the vehicle groups to determine if the methamphetamine prime and stress conditions used were capable of reinstating responding. All types of comparisons were considered statistically significant if p<0.05.

### Results

Grubb's Test analyses identified one rat in the 7.5 mg/kg AV411 stress-reinstatement group as an outlier (z=2.93) and its data were excluded from subsequent analyses. The number of active lever presses during the last day of self-administration were non-significantly different amongst the test groups, indicating that the rats had been trained to self-administer methamphetamine to similar levels prior to extinction training [F(2,32)=0.0356; p=0.5275] (data not shown). Mean (±SEM) active lever presses during the last session of extinction by the vehicle group was 20.08 (±3.10) and declined in the 2.5 mg/kg and 7.5 mg/kg AV411 test groups to 17.75 (±3.39) and 12.45 (±2.54) respectively. These differences in numbers of active lever presses during the last day of extinction, however, were non-significantly different [F(2,33)=1.591; p=0.2194] amongst the test groups. Mean (±SEM) number of active lever presses during the last session of extinction emitted by the vehicle treatment group was 20.08 (±3.10), and increased to 33.67 (±6.43) during the reinstatement test session which was a statistically significant increase (t=1.851, df=11, p=0.0456) indicating that footshock was able to effectively reinstate responding under the present conditions (see FIG. 1A).

### Stress Reinstatement Test

FIG. 12A shows mean numbers of active lever presses emitted during the reinstatement test session for each of the test groups. Pretreatment with 2.5 (q=2.401) and 7.5 mg/kg (q=2.645) AV411 significantly reduced (p<0.05, one-tailed comparisons) footshock-induced reinstatement relative to vehicle pretreatment (VEH). Inactive-lever presses (FIG. 12B) were uniformly low for all test groups and nonsignificantly different [F(2,32)=0.836; p=0.4428] during the reinstatement test session from one another.

### Prime Reinstatement Test

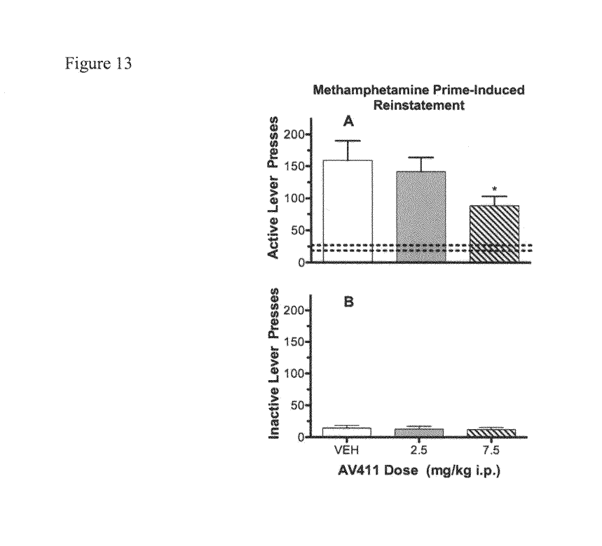

The mean numbers (±S.E.M.) of active lever presses during the last day of self-administration for the vehicle, 2.5 mg/kg AV411 an 7.5 mg/kg AV411 groups were 46.92 (±3.15), 64.83 (±10.73) 332 58.33 (±15.61), respectively, and were not-significantly different amongst the test groups indicating that the rats had been trained to self-administer methamphetamine to similar levels prior to extinction training [F(2,33)=0.6691; p=0.5190] (data not shown). Mean (±SEM) active lever presses during the last session of extinction by the vehicle group was 27.08 (±4.67), and declined in the 2.5 mg/kg and 7.5 mg/kg TDP 32,888 test groups to 20.92 (±5.57) and 18.67 (±13.05), respectively. These differences in numbers of active lever presses during the last day of extinction, however, were not statistically significant [F(2,33)=0.8500; p=0.4366] amongst the test groups. Mean (±SEM) number of active lever presses during the last session of extinction emitted by the vehicle treatment group was 27.08 (±4.67), and increased to 159.1 (±31.19) during the reinstatement test session which was a statistically significant increase (t=4.36, df=11, p=0.0006) indicating methamphetamine primes were able to effectively reinstate responding under the present conditions (see FIG. 13A).

FIG. 13A shows mean numbers of active lever presses emitted during the reinstatement test day for each of the test groups. Pretreatment with AV411 resulted in dose-dependent decreases in active lever-presses, and were significantly lower (p<0.05, one-tailed comparison) in the 7.5 mg/kg treatment group (q=2.111) relative to the vehicle-treatment group. Inactive-lever presses (FIG. 13B) were uniformly low for all test groups and non-significantly different [F(2,33)=0.061; p=0.9406] from one another.

The above results and accompanying data in FIGS. 12 and 13 indicate that stress-induction by footshock conditions used in this study effectively reinstated methamphetamine response in vehicle-treated rats previously reinforced with methamphetamine. The observations that the vehicle-treated rats in both the footshock and prime conditions emitted significantly more lever presses on the testday relative to their corresponding last day of extinction indicates that the experimental conditions used were appropriate for evaluating treatments which could reduce levels of reinstatement.

Although, Shepard et al., 2004 have published a report in which footshock has been used to reinstate responding previously reinforced with methamphetamine in laboratory animals, the experimental conditions for training, extinction, and reinstatement described above differ in many ways from those disclosed by Shepard. Methamphetamine priming conditions used in the present study also effectively reinstated response.

Thus, when AV411 was tested it reduced levels of stress-induced reinstatement at 2.5 and 7.5 mg/kg, and of prime-induced reinstatement at 7.5 mg/kg. Without being bound to any particular theory, the present inventors hypothesize that ibudilast's ability to attenuate stress- and prime-induced reinstatement stems from its ability to attenuate the activation of microglia and astroglia, and/or to inhibit certain PDE's—expecially PDE's-3,4,10,11, and/or to reduce the production of inflammatory cytokines such as TNFα and IL-1β, and/or to increase the production of various anti-inflammatory and nerve growth factors including IL-10 and GDNF.

Although preferred embodiments of the subject invention have been described in some detail, it is understood that obvious variations can be made without departing from the spirit and the scope of the invention as claimed herein. Thus, other psychostimulants selected from the group consisting of an amphetamine, a methylenedioxymethamphetamine, a methamphetamine, and a dextroamphetamine would also give similar results.

All references cited herein, including patents, patent applications and other publications, are hereby incorporated by reference in their entireties.

What is claimed is:

1. A method for suppressing the release of dopamine in the nucleus accumbens of a subject suffering from a psychostimulant addiction or psychostimulant dependence comprising administering to the subject an effective amount of ibudilast.

2. The method of claim 1, wherein the psychostimulant addiction or psychostimulant dependence is an addiction or dependence on a drug selected from the group consisting of an amphetamine, a methylenedioxymethamphetamine, a methamphetamine, and a dextroamphetamine.

3. The method of claim 1, wherein the psychostimulant addiction or psychostimulant dependence is an addiction or dependence to methamphetamine.

4. The method of claim 1, wherein the ibudilast is administered systemically or centrally.

5. The method of claim 1, wherein multiple therapeutically effective doses of ibudilast are administered to the subject according to a daily dosing regimen or intermittently.

6. A method for treating a psychostimulant addiction or psychostimulant dependence comprising administering to a subject in need thereof a therapeutically effective amount of ibudilast.

7. The method of claim 6, wherein the psychostimulant addiction or psychostimulant dependence is an addiction or dependence on a drug selected from the group consisting of an amphetamine, a methylenedioxymethamphetamine, a methamphetamine, and a dextroamphetamine.

8. The method of claim 7, wherein the psychostimulant addiction or psychostimulant dependence is to the drug methamphetamine.

9. The method of claim 8, wherein ibudilast diminishes or eliminates methamphetamine-related addiction or methamphetamine-related dependence behavior in said subject.

10. The method of claim 9, wherein ibudilast diminishes or eliminates methamphetamine-related addictive behavior cues in the subject.

11. The method of claim 6, further comprising administering to said subject ibudilast to diminish or eliminate symptoms of methamphetamine withdrawal syndrome.

12. The method of claim 6, wherein ibudilast diminishes or eliminates activation of glial cells, astrocytes, or microglia in the subject.

13. The method of claim 6, wherein ibudilast diminishes or eliminates drug-induced increases in interleukin-1 expression in the subject.

14. The method of claim 8, further comprising administering one or more agents other than ibudilast for treating an methamphetamine addiction.

15. The method of claim 7, wherein ibudilast inhibits in said subject methamphetamine-related dependence behavior selected from conditioned place preference, sensitization, tolerance, or craving.

16. A method for inhibiting relapse of psychostimulant addiction or dependence in a human subject comprising administering to human subject having a history of psychostimulant addiction or dependence an effective amount of ibudilast.

17. The method of claim 16, wherein the psychostimulant addiction or psychostimulant dependence is an addiction or dependence on a drug selected from the group consisting of an amphetamine, a methylenedioxymethamphetamine, a methamphetamine and a dextroamphetamine.

18. The method of claim 17, wherein the psychostimulant addiction or psychostimulant dependence is methamphetamine addiction or dependence.

19. The method of claim 18, wherein relapse is stress-induced, anxiety-induced, induced by an episodic exposure to methamphetamine, or withdrawal-induced.

20. The method of claim 16, wherein an effective amount of ibudilast ranges from about 10 mg to about 300 mg per day.

21. The method of claim 16, further comprising administering one or more agents other than ibudilast for treating stress-induced, anxiety-induced, induced by an episodic exposure to methamphetamine, or withdrawal-induced psychostimulant relapse.

22. A method for preventing relapse of psychostimulant addiction or dependence in a human subject having a history of psychostimulant addiction or dependence comprising administering to a human subject in need thereof an effective amount of ibudilast.

23. The method of claim 22, wherein the psychostimulant addiction or dependence is an addiction or dependence on a drug selected from the group consisting of an amphetamine, a methylenedioxymethamphetamine, a methamphetamine, and a dextroamphetamine.

24. The method of claim 23, wherein the psychostimulant addiction or dependence is a methamphetamine addiction or dependence.
